(19) United States
(12) Patent Application Publication
(10) Pub. No.: US 2005/0277934 A1
(43) Pub. Date: Dec. 15, 2005

(54) Rod delivery device and method

(76) Inventor: Arnold B. Vardiman, San Antonio, TX (US)

Correspondence Address: Malcolm E. Whittaker;Whittaker Law Firm, Suite 606, 8 Greenway Plaza, Houston, TX, 77046, US

(21) Appl. No.: 11/150,705

(22) Filed: Jun. 10, 2005

Related U.S. Application Data
(60) Provisional application No. 60/578,658, filed on Jun. 10, 2004.

(51) Int. Cl. A61B 17/70

(52) U.S. Cl. 606/61

(57) ABSTRACT

A minimally invasive spinal fixation system used for spinal arthrodesis or motion preservation spinal repair, comprising a plurality of pedicle screws, including a first screw placed into a first vertebral body, and a second screw placed into a second vertebral body; an attachment assembly for connecting said pedicle screws, said assembly comprising a connector for attaching to said first screw and said second screw; and, a removable guide for percutaneously attaching the connector to said first screw and said second screw.

### RELATED APPLICATION

This application claims priority to provisional application No. 60/578,658, filed Jun. 10, 2004.

Attorneys for Inventor: Malcolm E. Whittaker, Registered Patent Attorney No. 37,965, Whittaker Law Firm, 8 Greenway Plaza, Suite 606, Houston, Tex. 77046

### TECHNICAL FIELD OF THE INVENTION

The technical field of the invention relates to percutaneous rod delivery.

The present invention relates to a rod delivery device for percutaneous surgery.

The technical field of the invention relates to a method of percutaneous rod delivery.

The present invention relates to a method of delivering a rod during percutaneous surgery.

### BACKGROUND OF THE INVENTION

The bony elements of the spinal column are vulnerable to trauma, cancer and a variety of degenerative conditions that result in the loss of structural integrity of the bony spine. Any loss of structural integrity may have potentially catastrophic loss of neurological function or even paralysis.

Restoring the structural integrity of the spine depends on successful bony healing. Bony healing is also referred to as “bony fusion.” Bony healing is greatly improved by implanted devices that are internal “splints” that immobilize and strengthen the spine during bony healing.

Typically, these internal “splints” are implanted devices such as pedicle screws. These implanted devices, such as pedicle screws, are inserted posteriorly into the thoracic and lumbar spine and then attached to rods or plates to immobilize the spine and allow solid bony fusion.

Recent advances in surgical technique allow pedicle screws to be placed and rods implanted through very small skin incisions. These small incisions are typically referred to as “percutaneous” exposures.

Currently, pedicle screws interconnect with rods or plates. The pedicle screws are inserted posteriorly into the vertebrae of the thoracic and lumbar spine. A single rod is then passed through each of the multiple pedicle screws. Currently, one major rod delivery technique involves delivering a rod through a fixed arc. Conventional surgical methods are adequate when the rod has a fixed path of delivery, such as when the pedicle screws that have been inserted are well aligned. This current technique is inadequate for three reasons. First, because the rod cannot be directed safely around vital structures or bony obstructions. Second, the current technique also allows no choice in the contour of the rod to match the normal curvature of the spine. Third, the rod can only be delivered through a fixed arc. Because the rod can only be delivered through a fixed arc, this limits the ability to pass a rod between multiple pedicle screws when alignment of those screws is imperfect and also limits the length of the rod that can be delivered. The present invention addresses these problems and also continues to use percutaneous limited access techniques. Percutaneous techniques are desirable because pedicle screws are fixed with minimal tissue trauma, less pain and less wound related complications than an open surgical technique. As discussed above, current percutaneous techniques are insufficient when used over multiple pedicle screw segments or when pedicle screw placement is irregular or spinal curvatures do not match pre-determined rod curvature.

As discussed immediately above, the current major rod delivery technique involves delivering the rod using a fixed path of delivery. Usually, it is relatively uncomplicated to connect two points with a straight line. The concept of connecting two points with a straight line is the same principal that applies to interconnecting two pedicle screws with a rod.

When a surgeon must interconnect a series of more than two pedicle screws using a rod, the surgeon, typically, is required to locate the pedicle screws in the bony elements in order to minimize interference with bony structures and also avoiding neural structures. These bony structures and the requirement to avoid neural structures frequently prevent delivering a rod along the desired straight line between two pedicle screws. Using the current methods, the surgeon may have to locate the pedicle screw in the bony elements in a way that is less than ideal for minimizing interference with bony structures and avoiding neural structures in order to establish co-linearity, i.e. a straight line, between the pedicle screws. Put another way, the current methods force a surgeon to focus more on co-linearity of the pedicle screws and less on positioning the pedicle screw to minimize interference with bony structures and avoiding neural structures.

### SUMMARY OF THE INVENTION

The present invention provides a method for delivering a rod using a rod handle.

Therefore, in accordance with a basic aspect of the invention there is provided a handle and a rod. The handle is used to maneuver the rod though two or more implant devices, such as pedicle screws, that resulting in a construct that acts as an internal splint to help immobilize and strengthen the spine during the period of bony healing.

The present invention also provides for a handle; a bayonet attachment to the handle, and a rod. The handle is used to maneuver the rod though two or more implant devices, such as pedicle screws, that act as internal splints to help immobilize and strengthen the spine during a period of bony healing. The bayonet attachment, in cooperation with pedicle screw extenders, assists the surgeon in guiding the rod through the channels of the pedicle screws.

The present invention also provides for a handle, pedicle screws, screw extenders and a neuronavigational system using detectional spheres.

The present invention also provides for a steerable handle and a rod.

The present invention also provides for a pedicle screw with an adjustable channel section.

The present invention also provides for a multi-channeled pedicle screw.

The present invention also provides for alternative designs for a multi-channeled pedicle screw.

The present invention also provides for a pedicle screw with a loop.

The present invention also provides for a handle, a selection of various shafts and a selection of various rods.

The present invention also provides for various shafts. Each of the shafts is preferably selected to accommodate the size and shape of a variety of patient's bodies and also the preference of the surgeon.

The present invention also provides for various rods of different size, shape and geometry. Each of the rods is preferably selected to accommodate the geometry of the rod delivery path and the curvature of the segment of the spine to be immobilized.

The present invention also provides for various rods of different, size, shape and geometry. Each of the rods is preferably selected to minimize pathway divergence and avoid bony obstructions on the rod delivery path.

The present invention also provides for apparatus to placing a rod using a retrograde placement technique.

The present invention also provides for a rod that is flexible and may be selectively made rigid (i.e. hardened) after placement in the heads of two or more pedicle screws.

The present invention also provides for a method of performing percutaneous pedicle screw insertion.

The present invention also provides for a method of selecting appropriate pedicle screws.

The present invention also provides for a method of selecting an appropriate handle.

The present invention also provides for a method of selecting an appropriate rod.

The present invention also provides for a method of performing free-hand percutaneous rod insertion.

The present invention also provides for a method of performing percutaneous rod insertion using pedicle screw extenders.

The present invention also provides for a method of performing percutaneous rod insertion using neuronavigational techniques.

The present invention also provides for a method of performing percutaneous rod insertion using retrograde techniques.

The present invention also provides for a method of performing percutaneous rod insertion in conjunction with a steerable rod.

The present invention also provides for a minimally invasive spinal fixation system using spinal arthrodesis or motion preservation spinal repair with a plurality of screws placed into vertebral bodies, a attachment assembly for connecting the pedicle screws. The attachment assembly for connecting the pedicle screws with a connector and a removable guide for percutaneously attaching the connector to the pedicle screws.

The present invention also provides for a minimally invasive method of using pedicle screws to stabilize vertebral bodies anatomically positioned in a patient. The method having steps of percutaneously placing pedicle screws into vertebral bodies; percutaneously inserting a connector into the patient in a first position adjacent the first pedicle screw, with the connector designed to accommodate the anatomical positions of the vertebral bodies and the orientations of the first and second pedicle screws; guiding the connector from the first position to a second position adjacent the second pedicle screw; and, attaching the connector to the first pedicle screw and the second pedicle screw.

The present invention also provides for a surgical kit for minimally invasive spinal arthrodesis or motion preservation spinal repair with the kit having a plurality of pedicle screws; a plurality of connectors and a guide with a handle and a plurality of removable shafts attachable to the connectors; the shafts designed to connect one or more of the connectors.

The present invention also provides for motion preservation spinal repair such that a connector might be sufficiently flexible such as to allow some movement between the vertebral bodies that have been interconnected by the connector. It should be noted that the motion preservation is not inconsistent with arthrodesis (the rigid fusing of bone) because it may be desirable to allow some motion between vertebral bodies that have been interconnected. Some medical professionals also increasingly believe that using semi-flexible connectors between the interconnected vertebral bodies may allow motion preservation. This can be desirable because it allows some movement in the patient's spine. A semi-flexible connector, such as a thin “bendable” rod, a polymer rod or the like may satisfy this possible need for a semi-flexible connector. However, other medical professionals believe that arthrodesis is desirable because it provides for bony fusion and more effective bony and spinal healing. Both of these techniques, arthrodesis and motion preservation spinal repair, are within the scope of the present invention.

The present invention also provides for minimally invasive spinal fixation because it is intended that the surgery to apply the present invention is percutaneous surgery or a similar minimally invasive surgery.

These and other embodiments will be more fully appreciated from the description below.

### BRIEF DESCRIPTION OF THE DRAWINGS

FIG. 1 is a perspective view of an embodiment a rod delivery device with a handle, a shaft and a rod.

FIG. 2 is a side view of a sextant in use showing the current state of the technology.

FIG. 2A a top detailed view of the problems a surgeon may encounter when the tip of a rod contacts a bony obstruction on a vertebrae when using the sextant current state of the art technology shown at FIG. 2.

FIG. 2B is a top detailed view of the “pathway divergence” problem that a surgeon may encounter when the tip of the rod is obstructed by a bony obstruction when using the sextant current state of the art technology, shown at FIG. 2. The deflection off a bony obstruction or bony prominence alters the trajectory of the rod making it impossible to engage the pedicle screw and potentially directs the screw into vulnerable soft tissues, visceral or neural elements.

FIG. 2C is a side view of the use of the sextant current state of the technology in conjunction with a rod in both the concave (lower) and convex (upper) portions of the human spine.

FIG. 3 is another perspective view of an embodiment of the rod delivery device and of a handle, a shaft and a rod of the present invention.

FIG. 3A is a detailed view of a portion of FIG. 3 illustrating a type of connection between the shaft and the rod illustrated in FIG. 3.

FIG. 4 is a perspective view of an alternative embodiment of the rod delivery device and of a handle, a shaft and a rod of the present invention.

FIG. 4A is a detailed view of a portion of FIG. 4, illustrating a type of connection between the shaft and the rod illustrated in FIG. 4.

FIG. 5 is a side view of a handle, various alternative shafts and various alternative rods of the present invention.

FIG. 6 is a side view of the present invention in use in both the concave and convex portions of the human spine.

FIG. 6A is a top detailed view of the present invention illustrating the use of a rod pre-selected by the surgeon to avoid a bony obstruction.

FIG. 6B is a top detailed view of an alternative embodiment of the present invention illustrating the use of a rod pre-selected by the surgeon to avoid a bony obstruction.

FIG. 7 is a perspective view of an alternative embodiment of a rod delivery device, including a handle, a shaft, a bayonet attachment and a rod.

FIG. 7A is a cross-sectional view of the bayonet attachment illustrated in FIG. 7.

FIG. 7B is a perspective view of the method of using the bayonet attachment seen in FIG. 7.

FIG. 8 is a perspective view of a pedicle screw and a screw extender.

FIG. 9 is a perspective view of another alternative embodiment of a rod delivery device illustrating a handle, a shaft, a rod, pedicle screws, screw extenders and a neuronavigational system using detectional spheres.

FIG. 9A is a perspective view of a pedicle screw and a screw extender in conjunction with a detection sphere located on the top of the screw extender.

FIG. 9B is a conventional comparator device for comparing the position of detection spheres.

FIG. 9C is a conventional display that will use the information from the detection spheres and the comparator [FIG. 9B] to assist a surgeon in guiding the tip of the rod through the channels of the pedicle screws.

FIG. 9D is a perspective view of the method of using the rod delivery device of the present invention seen in FIGS. 9A, 9B and 9C.

FIG. 10 is a perspective view of another alternative embodiment the rod delivery device of the present invention including a handle and an alternative embodiment of a rod having a steerable tip.

FIG. 11 is a perspective view of adjustable uni-channeled pedicle screws, multi-channeled pedicle screws and rods of the present invention.

FIG. 11A is a perspective view of inserting a rod between the first and second pedicle screws seen in FIG. 11.

FIG. 11B is a perspective view of inserting a rod between the second and third pedicle screws seen in FIG. 11.

FIG. 11C is a perspective view of inserting a rod between the third and fourth pedicle screws seen in FIG. 11.

FIG. 11D is a perspective view of inserting a rod between the fourth and fifth pedicle screws seen in FIG. 11.

FIG. 12A is a perspective view of a series of pedicle screws of the present invention that have been fastened together using four rods of the present invention.

FIG. 12B is an alternative view of the present invention seen in FIG. 12A with certain portions of the vertebrae removed to allow a better view of the pedicle screws and rods of the present invention.

FIG. 13A is a side view of an alternative embodiment of the present invention including use of a retrograde rod and illustrating the step of inserting a pathfinder through a patient's skin and through the head of at least one pedicle screw.

FIG. 13B is a side view of the alternative embodiment seen in FIG. 13A and illustrating a handle, pathfinder and an embodiment of a flexible rod of the present invention.

FIG. 13C is a side view of the alternative embodiment seen in FIGS. 13A and 13B and illustrating positioning an embodiment of a flexible rod in pedicle screws of the present invention. FIG. 13C also illustrates fastening an injector to inject core material into the interior core of a flexible rod of the present invention.

FIG. 13D is a side view of the alternative embodiment seen in FIGS. 13A, 13B and 13C and illustrates using an injector to inject core material into a flexible rod of the present invention.

FIG. 13E is a side view of the alternative embodiment seen in FIGS. 13A, 13B, 13C and 13D and illustrates disengaging the injector as well as making the core material of the present invention rigid.

FIG. 13F is a perspective view of a possible interconnection, using a threaded lock, between the pathfinder and handle of the present invention, seen in FIG. 13A-13E.

FIG. 13G is a perspective view of a possible interconnection, using a snap-on lock. between the pathfinder and handle of the present invention, seen in FIGS. 13A-13E.

FIG. 14 is a side view of alternative embodiments of the flexible rod of the present invention.

FIG. 15 is a perspective view of an alternative embodiment of the present invention illustrating a rod and a flexible rod device.

FIG. 16 is another perspective view of a handle and a flexible rod, as seen in FIG. 15, in use.

FIG. 16A is a perspective view of the flexible rod device seen in FIGS. 15 and 16.

FIG. 17 is a perspective view of an alternative loop pedicle screw of the present invention.

FIG. 18A is a front view of an embodiment of an adjustable channel section of an adjustable uni-channeled pedicle screw of the present invention.

FIG. 18B is a side view of the embodiment illustrated in FIG. 18A of the adjustable channel section of the adjustable uni-channeled pedicle screw of the present invention.

FIG. 18C is a side view of the embodiment seen in FIGS. 18A and 18B illustrating how the channel portion of the adjustable uni-channeled pedicle screw may be adjusted.

FIG. 19A is a side view of an embodiment of an adjustable uni-channeled pedicle screw of the present invention.

FIG. 19B is a front view of the embodiment of the adjustable uni-channeled pedicle screw of the present invention seen in FIG. 19A.

FIG. 20 is a front view of an embodiment of a multi-channeled pedicle screw of the present invention.

FIG. 21 is a front view of an embodiment of the multi-channeled pedicle screw of the present invention.

FIG. 22 is a front view of an alternative embodiment of the multi-channeled pedicle screw of the present invention.

FIG. 23 is a front view of an alternative embodiment of the multi-channeled pedicle screw of the present invention.

### DESCRIPTION OF THE PREFERRED EMBODIMENTS

Corresponding reference numbers indicate corresponding parts throughout the several views of the drawings and specification.

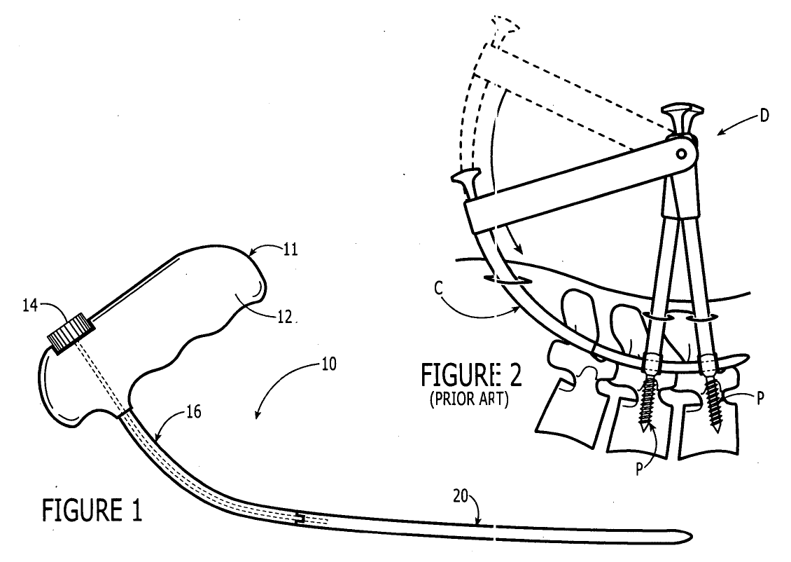

FIG. 1 illustrates an embodiment of the rod delivery device 10 of the present invention. Rod delivery device 10 includes a handle 11, a shaft 16 and a rod 20.

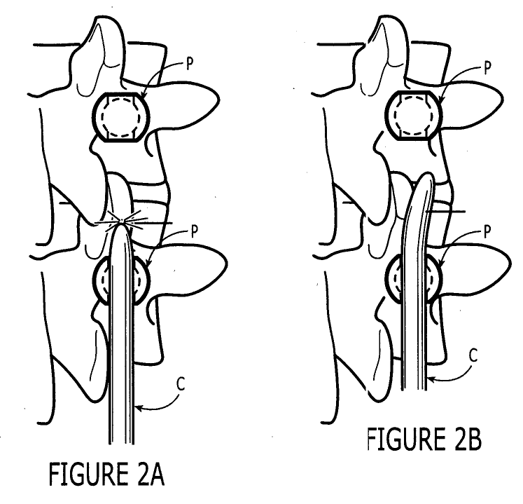

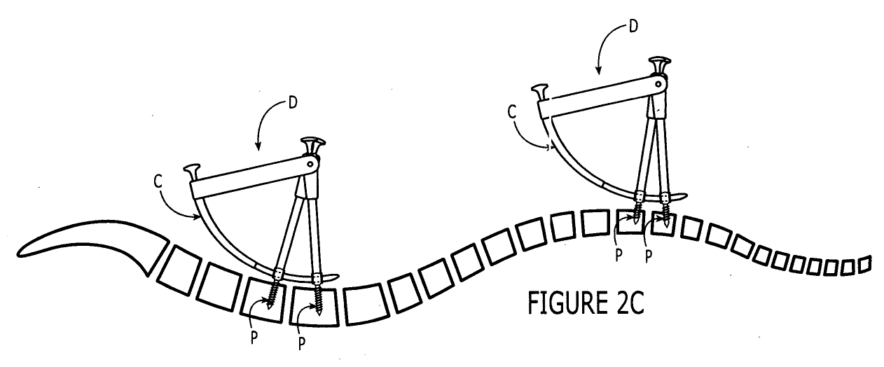

FIG. 2 illustrates a sextant type rod delivery device that primarily employs a rod delivery mechanism that delivers a fixation rod via a fixed trajectory along a fixed arc. While these “sextant” type devices are commonly used today and are highly successful for limited numbers of fixation points, they are of limited value for multiple fixation points and also when the contours of the human spine do not mirror the contour of the fixation rod the surgeon is attempting to use to fixedly connect pedicle screws using fixation rods. The sextant type rod delivery device D releasably holds a curved fixation rod C, also known as a rod, and delivers the rod by sweeping the rod C through an arc which is parallel to curved fixation rod C's path. After the surgeon has configured the sextant D to deliver the rod C along the desired arc, the rod C will not vary from that path. While this may be desirable for certain situations, it is highly undesirable when the rod C encounters one or more bony obstructions.

FIG. 2A illustrates an example a problem associated with a “fixed arc” sextant type rod delivery device D. Namely, because the rod C is delivered in a predetermined path that is fixed and cannot be “steered” around obstacles such as bony obstruction because the tip of rod C has collided with a bony obstruction. At best, the surgeon will likely have to withdraw rod C and raise the pedicle screws P to bring the rod C above the bony obstruction. Of course, this is undesirable because this means that the pedicle screw P is more shallowly implanted in the bone of the vertebrae and therefore is less securely implanted into the bone. A dangerous consequence of colliding with a bony obstruction is that the bony obstruction will break off and the patient will suffer neurological damage. Of course, an unattached broken piece of bone is also undesirable and may require additional surgery to remove it. Both of these problems are undesirable.

FIG. 2B illustrates another example of a problem associated with a fixed arc sextant type rod delivery device D. Namely, because the rod C is delivered in a predetermined path, if the tip of rod C collides with a bony obstruction and the rod C is diverted away from its intended path, a condition known as “pathway divergence,” the rod C may veer off course and penetrate unintended areas. For example, the rod C could divert from its intended path and intrude into lung or liver tissue. Obviously, this highly undesirable. Also, in the upper spine, it is also possible for a rod C to travel underneath a rib and thereby intrude into the lung. Again, this is also highly undesirable.

FIG. 2C illustrates using a conventional sextant type rod delivery device D in both the lower (concave) and upper (convex) sections of the human spine. As can be seen on the sextant shown on the left side of FIG. 2C, illustrating use of a sextant type rod delivery device D, the curved rod C delivered by the sextant D roughly conforms to the contours of the patient's spine because both the rod and the spine are concave. However, FIG. 2C also shows that a conventional sextant type rod delivery device D is less appropriate when used in the upper (convex) portion of the human spine because of the convex shape of the human spine. The present invention substantially avoids this problem because it does not deliver a rod in a fixed arc.

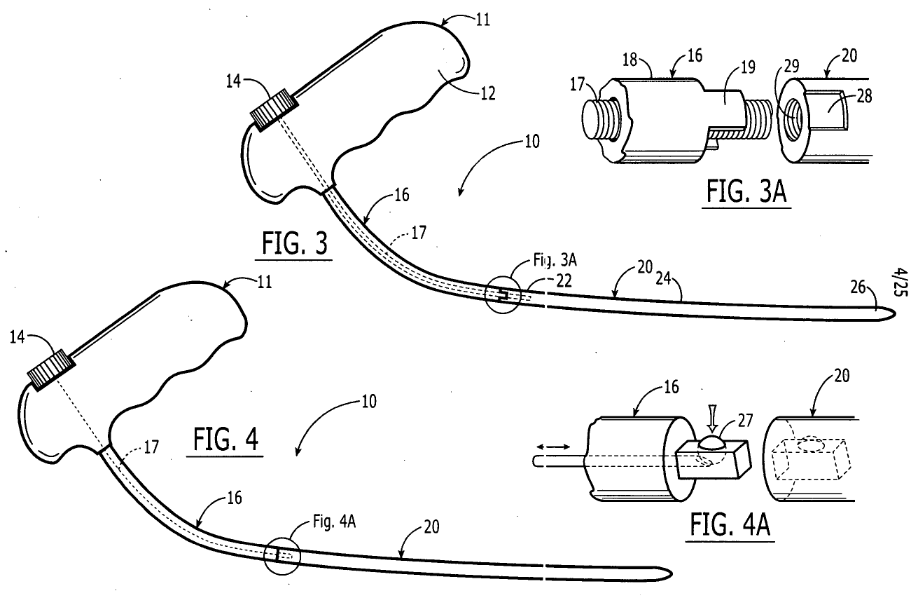

FIG. 3 illustrates a rod delivery device 10. Rod delivery device 10 includes a handle 11, a shaft 16 and a rod 20. Handle 11 includes a grip 12, release button 14, a connecting end 18, and a tongue 19. Grip 12 is shaped to maximize the controllability of handle 11, or as required by different circumstances, or the personal preference of the surgeon, different shaped grips 12 may be used. Rod 20 includes a proximal end 22, medial section 24, a distal end/tip 26, a groove 28 and an opening 29. Shaft 16 releasably interconnects handle 11 and rod 20.

Release button 14 is fastened to handle 11 such that as release button 14 is rotated, screw 17 also rotates and will screw into and out of a bore or opening 29. Tongue 19 is a part of a “tongue-in-groove” type connection. This “tongue-in-groove” interconnection is clearly seen at FIG. 3A. The tongue 19 and groove 28 substantially prevent rod 20 from rotating or spinning. Screw 17 is received by an opening 29. In the embodiment seen in FIG. 3A, opening 29 and screw 17 are threaded. As seen in FIG. 3A, the threaded portions of screw 17 and the threaded opening 29 mate rigidly to releasably fasten shaft 16 to rod 20. Collectively, tongue 19, groove 28, screw 17 and opening 29 substantially prevent rod 20 from rotating relative to handle 11 and shaft 16. Movement of rod 20 relative to handle 11 and shaft 16 is undesirable because it makes it more difficult for a surgeon to guide rod 20 into the patient and between pedicle screws. Relative movement between handle 11 and shaft 16 is also undesirable because it also makes it more difficult for a surgeon to guide rod 20 into the patient and between pedicle screws.

As shown in FIGS. 4 and 4A, shaft 16 can use a number of connection means to fasten to rod 20. For example, a “snap-lock” type device 27 is appropriate. Snap-lock type devices 27 are well known and are used for the purpose of illustrating an alternative type connection for shaft 16 and rod 20. Again, it is highly desirable to minimize or prevent rod 20 from rotating or moving relative to handle 11 or shaft 16.

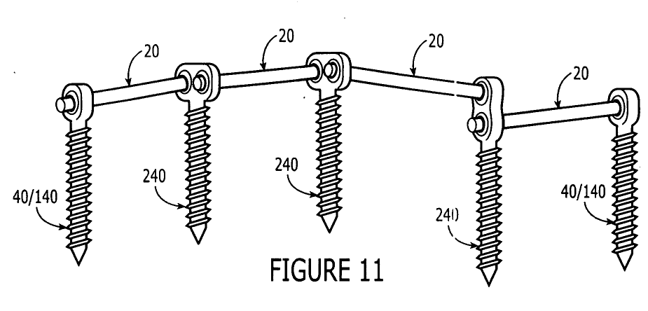

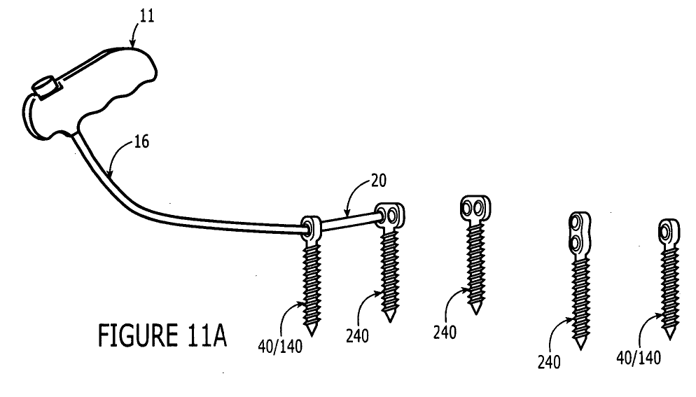

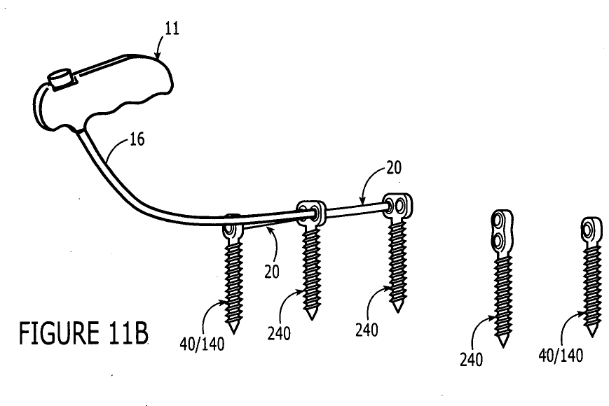

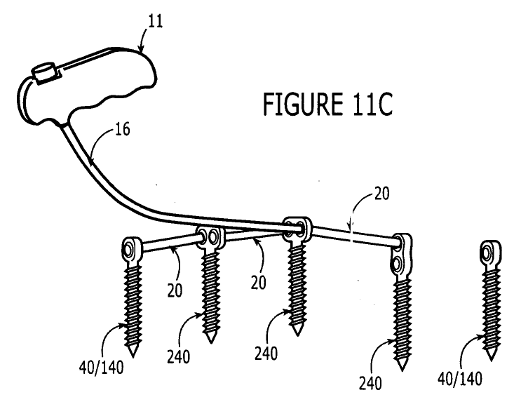

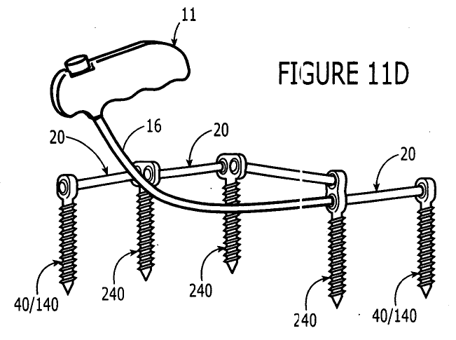

After rod 20 is fastened to handle 11 and shaft 16, a surgeon will use handle 11 to maneuver rod 20 through two or more implant devices, such as pedicle screws, that act as internal splints to help immobilize and strengthen the spine during a period of bony healing and fusion. Using this “free-hand,” and equivalent methods, handle 11 and shaft 16 serve as “removable guides.” Examples of pedicle screws as implant devices are seen at FIGS. 11, 12A, 12B, 17, 18A, 18B, 18C, 19A, 19B, 20, 21, 22 and 23.

As discussed above, handle 11 is fastened to shaft 16. Preferably, this is a releasable connection. Handle 11 and shaft 16 can use a number of connection means to connect. For example, a “snap-lock” type device is appropriate. Snap-lock type devices are well known. Also, a “tongue-in-groove” type connection is also appropriate. It is also within the scope of the present invention that handle 11 and shaft 16 could be rigidly and fixedly fastened. However, it is preferable to make shaft 16 releasably connected to handle 11. Again, it is highly desirable to minimize relative motion between handle 11 and shaft 16.

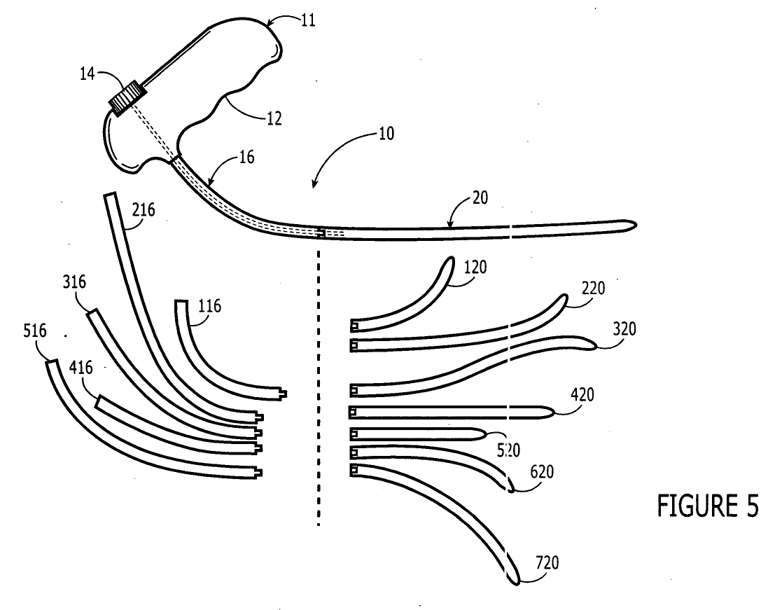

FIG. 5 shows the rod delivery device 10, also seen in FIG. 1. As seen in FIG. 5, both shaft 16 and rod 20 are interchangeable with alternative shafts and rods. A substantial advantage of the rod delivery device 10 is that a surgeon can select and interchange shaft 16 and rod 20 with an alternative shaft 116 or alternative rod 120. Typically, these alternative shaft 116 or alternative rod 120 are selected to offer more appropriate matching to the patient's body contours. A surgeon could also substitute alternative shafts 216, 316, 416 or 516 for shaft 16. Similarly, a surgeon could substitute alternative rods 220, 320, 420, 520, 620 or 720 for rod 20. The alternative shafts and rods seen in FIG. 5 are by way of example and not limitation. Any shape and configuration of shaft and rod that a surgeon might require could be fabricated. Typically, a surgeon will use an alternative rod or alternative shaft because of the configuration of the pedicle screws in the patient's spine, the configuration of the patient's spine or other anatomy, the presence of bony obstructions or other situations the surgeon may encounter when immobilizing a patient's spine using implant devices such as pedicle screws.

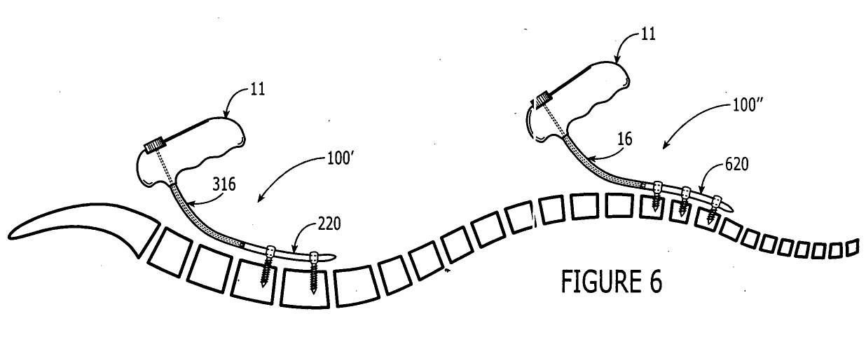

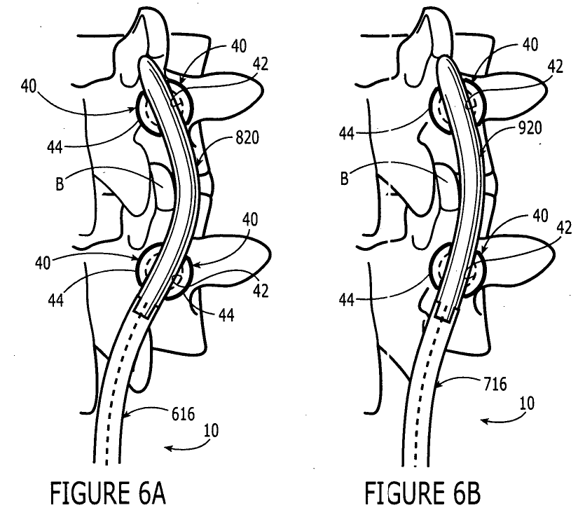

For example, as seen in FIG. 5, a surgeon might select alternative shaft 116 if the patient requiring surgery was slim and there was a thin layer of muscle and fascia located above the patient's spinal column. A surgeon might use alternative shaft 216 if the patient requiring surgery was obese and there was a thick layer of muscle and fascia located above the patient's spinal column. A surgeon might use alternative shaft 316 if the curvature of the patient's spine is shallow, such as seen in the rod delivery device 10 on the left in FIG. 6, in a convex portion of the spine. A surgeon might use alternative shaft 416 under other circumstances. A surgeon might use alternative shaft 516 to accommodate different spinal curvature and different patient muscle and fascia thickness.

Alternative rods 120, 220, 320, 420, 520, 620 and 720 are interchangeable with rod 20. For example, a surgeon might select alternative rod 120 to interconnect multiple pedicle screws if the pedicle screws were located on a section of the human spine where the curvature changes from convex to concave. A surgeon might select alternative rod 220 to interconnect multiple pedicle screws positioned in a section of the spine with a similar curvature to the shape of alternative rod 220, i.e. matching a concave section of the human spine. Similar principles apply with respect to alternative rod 320. A surgeon might select alternative rod 420, which is shorter than rod 20, to interconnect multiple pedicle screws that are located close together. A surgeon might also select alternative rod 520 to interconnect multiple pedicle screws that are positioned very close to one another. A surgeon might select alternative rod 620 to interconnect multiple pedicle screws located in a shallowly convex portion of the human spine. A surgeon might select alternative rod 720 to interconnect multiple pedicle screws in a steeply convex section of the human spine.

The alternative shafts and alternative rods seen in FIG. 5 are by way of example only. A surgeon could select an alternative shaft and alternative rod of different length, width, curvature and diameter as needed to interconnect multiple pedicle screws located in various sections of the human spine. The diameter of any of the alternative rods could also be selected based on the size of the orifice located in the pedicle screw. Generally, it is preferable to use a rod that is snuggly received by the channel of the head of the pedicle screw. As noted above these handles and shafts of the present invention serve as removable guides.

It is also within the scope of the present invention that a surgeon performing minimally invasive spinal surgery could use an attachment assembly with at least one connector, for attaching pedicle screws, and a removable guide. It is within the scope of the present invention that the attachment assembly that could be used to percutaneously connect pedicle screws could be rods, plates, pins, polymer or cement fillable-to-harden flexible rods, a link and insert flexible rod that can be stiffened using a tightener or a rod made of ferroelectric material that is pliable until exposed to electric current. These or equivalent attachment assemblies could be used with any of the devices or methods, or the equivalents, of the present invention.

FIG. 6 shows rod delivery device 100′ and a rod delivery device 100″ interconnecting multiple pedicle screws that have been implanted in a human spine. Typically, a surgeon will only use a single rod delivery device 10 at a time. However, FIG. 6 shows two rod delivery devices for the purpose of illustrating how a surgeon might use a variety of alternative shafts and alternative rods to interconnect multiple pedicle screws that have been implanted in a human spine.

Rod delivery device 100′ uses handle 11, alternative shaft 316 and alternative rod 220. Of course, as discussed above, a surgeon could choose another alternative shaft and another alternative rod. However, by way of example and not limitation, the surgeon has selected alternative shaft 316 and alternative rod 220 for the conditions seen in FIG. 6 with rod delivery device 100′.

Rod delivery device 100″ uses handle 11, shaft 16 and alternative rod 620. Of course, as discussed above, a surgeon could choose another alternative shaft and another alternative rod. However, by way of example and not limitation, the surgeon has selected shaft 16 and alternative rod 620 for the conditions seen in FIG. 6 with rod delivery device 100″.

FIG. 6A shows a rod delivery device 10 in use interconnecting two pedicle screws 40 that have been implanted in a human spine. As can be seen in FIG. 6A, alternative rod 820 is releasably connected to alternative shaft 616. A surgeon uses alternative shaft 616 and handle 11 [not seen] to drive alternative rod 820 through the channels 42 in the heads 44 of pedicle screws 40. As seen in FIG. 6A, a surgeon will select alternative rod 820 such that its geometry most smoothly allows alternative rod 820 to interconnect the two pedicle screws 40 seen in FIG. 6A so that alternative rod 820 does not collide with any bony obstructions B. Similarly, a surgeon will select alternative shaft 616 such that its geometry most smoothly allows alternative shaft 616 to drive alternative rod 820 so that alternative rod 820 does not collide with any bony obstructions B.

FIG. 6B shows a rod delivery device 10 in use interconnecting two pedicle screws 40 that have been implanted in a human spine. As can be seen in FIG. 6B, alternative rod 920 is releasably connected to alternative shaft 716. A surgeon uses alternative shaft 716 and handle 11 [not seen] to drive alternative rod 920 through the channels 42 in the heads 44 of pedicle screws 40. As seen in FIG. 6B, a surgeon will select alternative rod 920 such that its geometry most smoothly allows alternative rod 920 to interconnect the two pedicle screws 40 seen in FIG. 6B so that alternative rod 920 does not collide with any bony obstructions B. Similarly, a surgeon will select alternative shaft 716 such that its geometry most smoothly allows alternative shaft 716 to drive alternative rod 920 so that alternative rod 920 does not collide with any bony obstructions B. Of course, bony obstructions B do not always appear at consistent locations on the human spine. As such, a surgeon will select a handle 11 [not seen] that most readily allows him to drive a shaft and a rod smoothly to interconnect two or more pedicle screws 40. Of course, different bone configurations, the presence or absence of bony obstructions, the locations of the pedicle screws and other criteria are all factors which will influence a surgeon's decision as to which handle, shaft and rod to select to interconnect multiple pedicle screws. Because of the variety of geometries, there is no single “ideal” rod delivery device 10 for all situations. In fact, one of the advantages of the present invention is that it allows a surgeon to select from a variety of handles, shafts and rods to most smoothly interconnect multiple pedicle screws while minimizing or avoiding undesirable contact with neural structures and soft tissue or collisions with bony obstructions. In effect, the interchangeability of the handle, shaft and rod of the present invention allow a surgeon to select the “ideal” rod delivery device for interconnecting pedicle screws for a variety of situations.

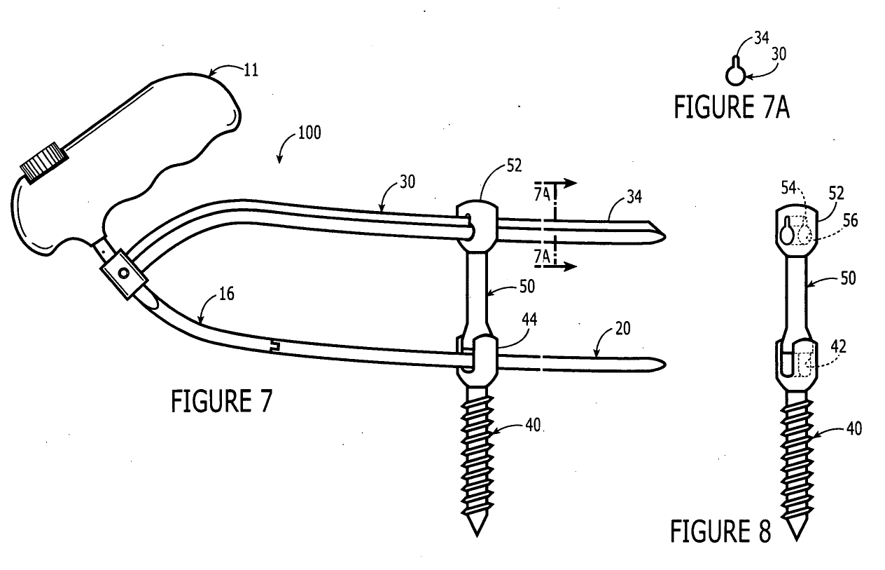

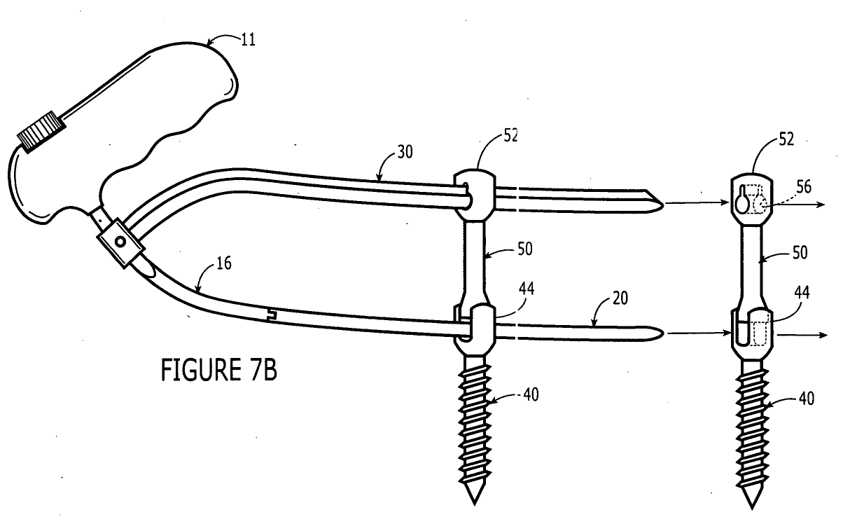

As shown in FIG. 7, an alternative embodiment of rod delivery device 10 provides for an alternative rod delivery device 100. Rod delivery device 100 includes a handle 11, shaft 16, rod 20 and a bayonet attachment 30. Handle 11 is used to maneuver rod 20 though two or more implant devices, such as pedicle screws 40, which act as internal splints to help immobilize and strengthen the spine during the period of bony healing. The bayonet attachment 30, in cooperation with pedicle screw extenders 50, assists the surgeon in guiding rod 20 through channels 42 of pedicle screws 40. In this embodiment of the present invention, the handle 11, shaft 16 and bayonet attachment serve as removable guides.

As shown in FIGS. 7, 7A, 7B and 8, screw extenders 50 act as guidance phantoms and also allow dynamic forces to be placed on the spine during insertion and tightening. As seen in FIG. 7, pedicle screw 40 is can be inserted posteriorly into the thoracic or lumbar spine. Screw extender 50 is removably fastened to pedicle screw 40. Because pedicle screw 40 is implanted into a vertebra, it is below the surface of the patient's skin. Screw extender 50 extends from the top 44 of pedicle screw 40, through the patient's skin, and is exposed to the surgeon above the patient's back. Head 52, of screw extender 50, includes notch 54 and groove 56. Notch 54 and groove 56 slidably receive bayonet attachment 30 and ridge 34. In a preferred embodiment, seen in FIGS. 7 and 7A, ridge 34 is slidably received by notch 54. Cooperatively, ridge 34 and notch 54 prevent bayonet attachment 30 from rotating relative to head 52 of screw extender 50. Effectively, screw extender 50's head 52 is an above skin phantom that is used to guide rod 20 through channels 42 of pedicle screws 40.

As shown in FIG. 7B, because rod 20 and bayonet attachment 30 move in tandem, when a surgeon guides bayonet attachment 30 through groove 56 and notch 54, rod 20 passes through channel 42 of pedicle screw 40.

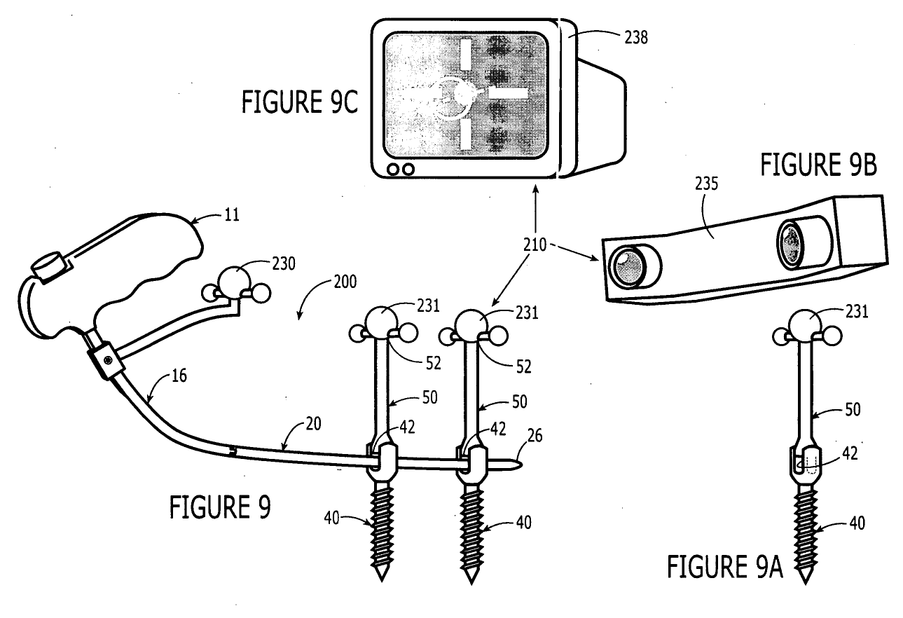

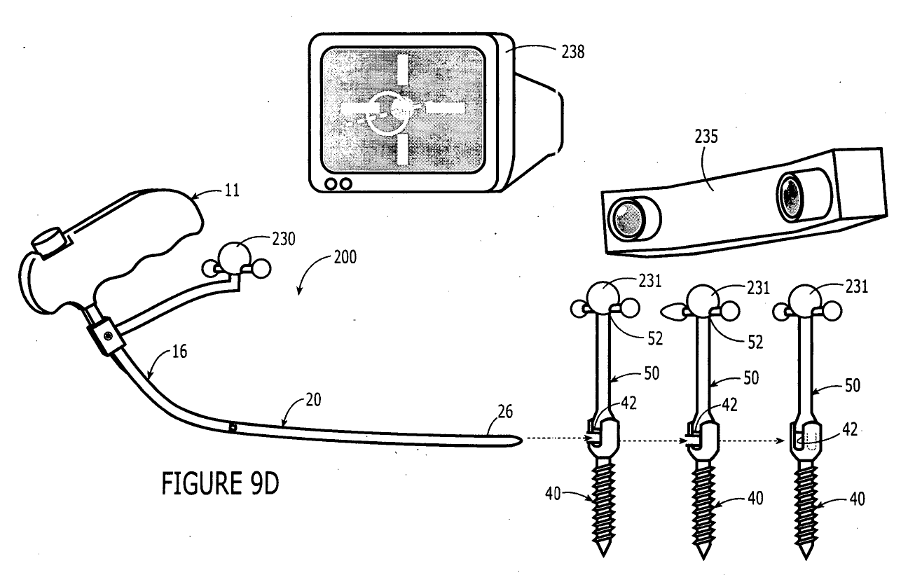

As seen in FIGS. 9, 9A, 9B, 9C and 9D, an alternative embodiment also provides for an alternative rod delivery device 200. Alternative rod delivery device 200 includes handle 11, shaft 16, rod 20, pedicle screws 40, screw extenders 50 and a neuronavigational system 210. Neuronavigational system 210 uses detectional spheres 230 and 231, comparator 235 and display 238.

Preferably, detectional spheres 231 are positioned on the head 52 of each screw extender 50 and detectional sphere 230 is positioned proximate to handle 11. It is important that detectional spheres 231 are fixedly positioned relative to screw extenders 50. It is also desirable that detectional sphere 230 remains in the same relative position to handle 11. If the detectional spheres do not remain fixed relative to these structures, the neuronavigational system cannot guide rod 20 through channel 42 of pedicle screw 40. Comparator 235 calculates the relative positions of handle 11, shaft 16, rod 20 and channels 42 of pedicle screw 40 because the relative positions of detector spheres 230 and 231 are known. Because comparator 235 “detects” the relative positions of handle 11, shaft 16, rod 20 and channel 42 of pedicle screw 40, display 238 visually displays this position information. The position information seen on display 238 indicates which direction a surgeon should move tip 26 of rod 20 to pass through the channels 42 of pedicle screws 40. Other than directional spheres 230 and 231, comparator 235 and display 238, the neuronavigational system 210 is not shown.

Neuronavigational systems, such as neuronavigational system 210, for spine and brain surgery are known and regularly used. For example, as disclosed at U.S. Pat. No. 5,383,454, issued to Buchholz, on Jan. 24, 1995, for system for indicating the position of a surgical probe within a head on an image of the head and at U.S. Pat. No. 6,236,875, issued to Buchholz, on May 22, 2001, for surgical navigation systems including reference and localization frames. Neuronavigational systems 210, and equivalents, are also known as “Computer Aided Surgery” Devices. It is within the scope of the present invention that a variety of Computer Aided Surgery Devices could act as removable guides for percutaneously attaching connectors, such as pedicle screws.

FIG. 9D shows a surgeon using alternative rod delivery device 200 to interconnect three pedicle screws 40. A surgeon uses neuronavigational system 210 to pass rod 20 through each of the three pedicle screws 40 seen in FIG. 9D. In this embodiment of the present invention, handle 11, shaft 16, rod 20, screw extenders 50 and neuronavigational system 210 serve as removable guides.

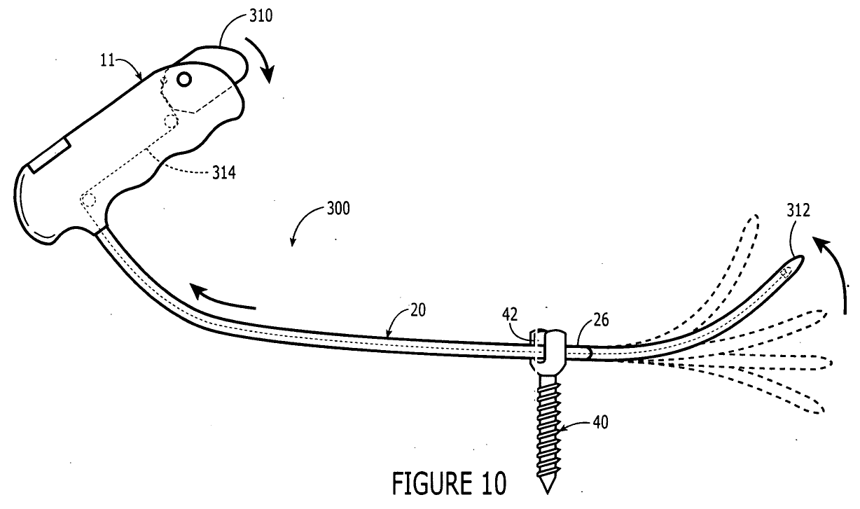

FIG. 10 illustrates another alternative embodiment of a rod delivery device. Steerable rod delivery device 300 includes handle 11, steering mechanism 310, rod 20, steerable rod tip 312, pedicle screw 40 and pedicle screw channel 42. A steerable rod tip 312 is fastened to the distal end 26 of rod 20. Steering wire 314 may be a wire, or other similar structure, that can guide steerable tip 312. Steerable rod delivery device 300 guides rod 20 through the channels 42 of multiple pedicle screws 40. While only one pedicle screw 40 is shown in FIG. 10, steerable rod delivery device 300 could guide rod 20 through multiple pedicle screws 40. In this embodiment of the present invention, handle 11, steering mechanism 310 and steerable rod tip 312 serve as removable guides.

Steerable devices, and particularly steerable catheters, are known to those skilled in the art. An example of a steerable device is a “shapeable handle for steerable electrode catheter” that is disclosed at U.S. Pat. No. 5,397,304, issued to Truckai, on Mar. 14, 1995.

FIG. 11 shows a series of rods 20 in conjunction with both pedicle screws 40/140 and multi-channeled pedicle screws 240. As can been seen in FIG. 11, it should be apparent that pedicle screws 40/140/240 can interconnect using rods 20 in a variety of configurations and geometries. The configuration shown is by way of example only. Pedicle screws 40/140/240 are alternative embodiments of pedicle screws.

FIG. 11A shows using a rod delivery device 10 to interconnect pedicle screw 40/140 to pedicle screw 240. In other words, interconnecting the first and second pedicle screws in the series of five seen in FIGS. 11A-11D, using a rod 20.

FIG. 11B shows using a rod delivery device 10 to interconnect pedicle screw 240 to pedicle screw 240. In other words, interconnecting the second and third pedicle screws in the series of five seen in FIGS. 11A-11B, using a rod 20.

FIG. 11C shows using a rod delivery device 10 to interconnect pedicle screw 240 to pedicle screw 240. In other words, interconnecting the third and fourth pedicle screws in the series of five seen in FIGS. 11A-11D, using a rod 20.

FIG. 11D shows using a rod delivery device 10 to interconnect pedicle screw 240 to pedicle screw 40/140. In other words, interconnecting the fourth and fifth pedicle screws in the series of five seen in FIGS. 11A-11D, using a rod 20.

After any of the pedicle screws seen in FIGS. 11A-11D are interconnected using rod delivery device 10, handle 11 and shaft 16 are withdrawn. Rod 20 remains between the pedicle screws for the purpose of interconnecting them. At that point, a surgeon might select a different shaft and a different rod in order to more smoothly interconnect the next pedicle screws. Of course, a surgeon could interconnect more than two pedicle screws in a single pass. In the alternative, the surgeon could choose to interconnect only two pedicle screws in a single pass. A surgeon is also not required to use a different handle 11 or shaft 16 with each rod insertion. However, one of the principle advantages of the present invention is that a surgeon can use a single rod to interconnect two or more pedicle screws. Another principle advantage is that a surgeon can readily select the most appropriate handle, shaft and rod needed to insert a pedicle screw to interconnect multiple pedicle screws. It is also within the scope ot the present invention that any of the alternative embodiments of the rod delivery device could be used to interconnect multiple pedicle screws. A surgeon is not limited to using just one rod delivery device. For example, a surgeon could use rod delivery device 10 to interconnect the first and second pedicle screws [seen in FIG. 11A] and use steerable rod delivery device 300 to interconnect the second and third pedicle screws [seen at FIG. 11B]. As seen in FIGS. 11A-11D, rod delivery device 10 uses shaft 16 and four rods 20 to interconnect all five pedicle screws. However, assuming it was appropriate, a surgeon could use an alternative rod to interconnect the pedicle screws seen in FIGS. 11A-11D. Also, as seen in FIGS. 11A-11D, rod delivery device 10 uses shaft 16 to interconnect all five pedicle screws. However, assuming it was appropriate a surgeon could use an alternative shaft. Also, again assuming it was appropriate, a surgeon could use a longer rod to interconnect three or more pedicle screws.

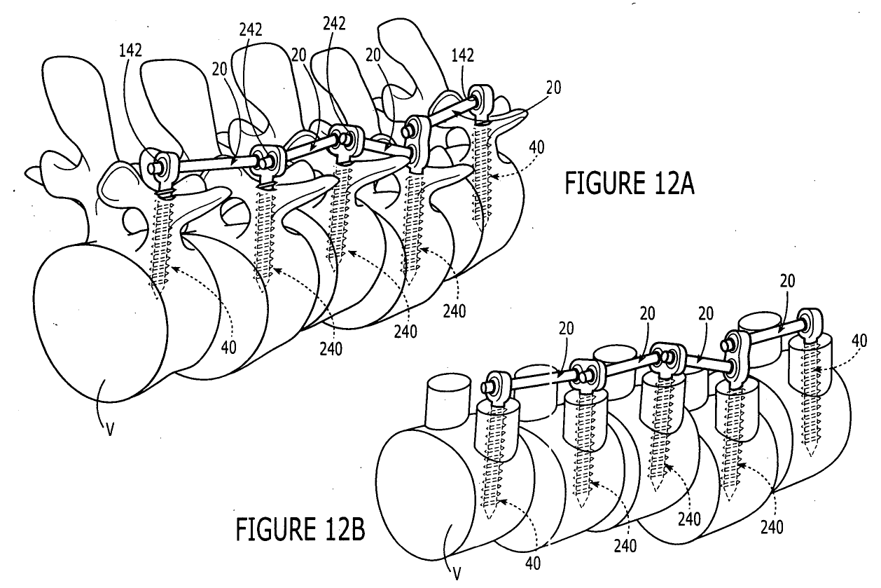

FIG. 12A shows pedicle screws 40 and 240 implanted into vertebrae V. In addition, FIG. 12A shows rods 20 interconnecting these pedicle screws 40/240. Of course, if a surgeon chose to select an alternative rod, the surgeon could use an alternative rod, to avoid a bony obstructions, soft tissue or neural tissue. Ideally, a surgeon would choose an alternative rod with a geometry that is configured to best avoid a bony obstruction, soft tissue or neural tissue. FIG. 12A only shows straight rods 20, however, straight rods may or may not be ideal depending on the geometry of the vertebrae V, the presence of bony obstructions, soft tissue or neural tissue. As also seen in FIG. 12A, both uni-channel pedicle screws 40 and multi-channel pedicle screws 240 may include adjustable channels 142/242. In the situation were a surgeon chooses to use a pedicle screw with an adjustable channel, there may be less need for alternative rods to accommodate the geometries necessary to interconnect the pedicle screws because the direction of the rod can be adjusted to face the rod more directly towards the channel of the next pedicle screw.

FIG. 12B is the same view as seen in FIG. 12A, with the exception that the upper portions of the vertebrae V have been removed to allow a better view of pedicle screws 40/240 and rods 20.

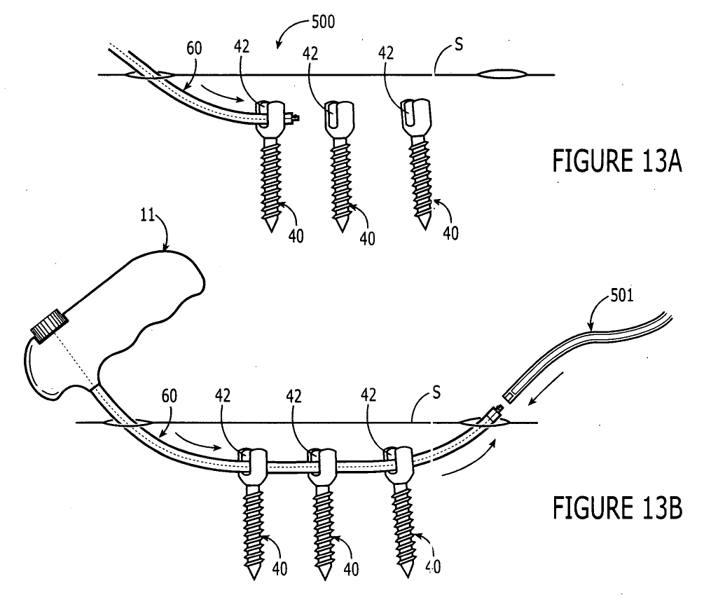

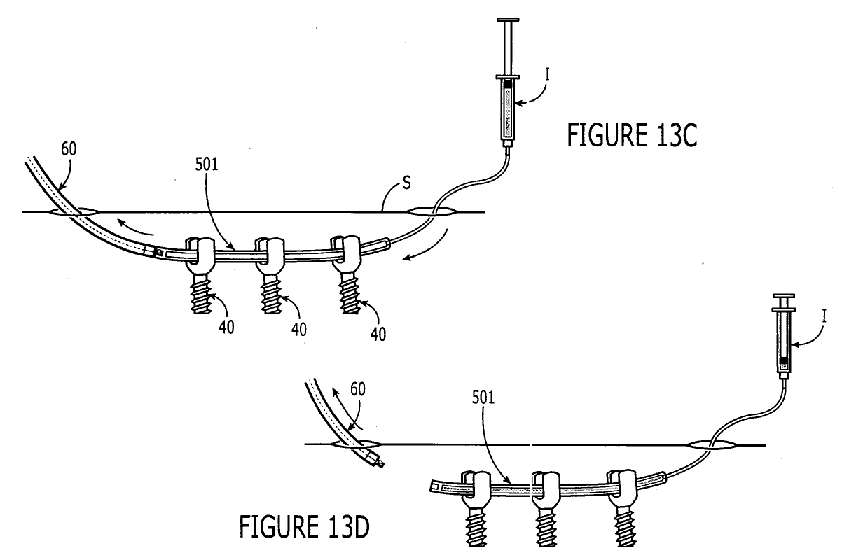

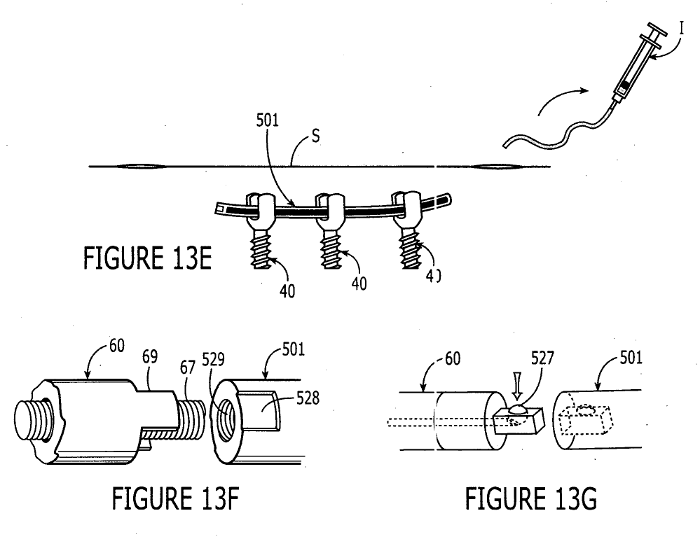

FIGS. 13A and 13B show a retrograde rod delivery device 500. Retrograde rod delivery device 500 includes handle 11, pathfinder 60 and flexible rod 501.

FIGS. 13A and 13B show a retrograde rod delivery device 500 being inserted through channels 42 of pedicle screws 40 using a handle 11 and a pathfinder 60.

FIG. 13B shows flexible rod 501 being releasably attached to the distal tip of pathfinder 60. The embodiment of flexible rod 501 seen in FIG. 13B is a hollow rod. Before flexible rod 501 is pulled below the patient's skin S, injector I is releasably connected such that injector I is in fluid communication with flexible rod 501.

FIG. 13C shows pathfinder 60 moving in a retrograde motion (i.e. being withdrawn to the left). Because pathfinder 60 moves in a retrograde motion, flexible rod 501 is positioned as seen in FIG. 13D. Preferably, flexible rod 501 should be positioned such that it interconnects multiple pedicle screws 40. As seen in FIG. 13D, injector I is still releasably connected to flexible rod 501 and is also in fluid communication. As seen in FIG. 13D, injector I injects a hardenable substance into flexible rod 501. For example, injector I could inject an epoxy into flexible rod 501. The hardenable substance is allowed to become rigid. As seen in FIGS. 13D and 13E, pathfinder 60 and injector I are preferably withdrawn after the hardenable substance becomes rigid.

FIG. 13E shows rod 501 acting as a rigid rod that serves as an internal splint that immobilizes and strengthens the spine during bony healing and fusion.

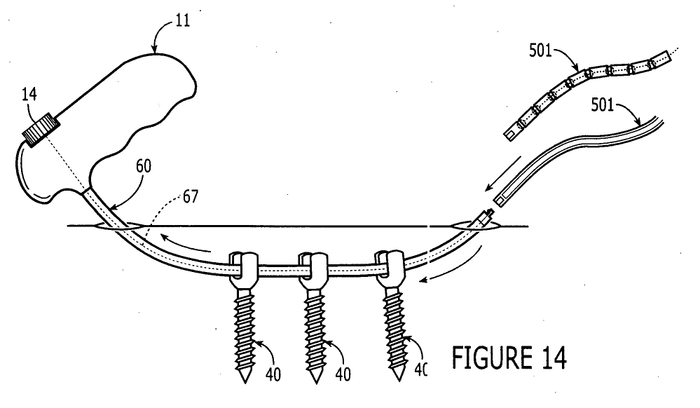

FIGS. 13F and 13G show different connectors and the associated apparatus to releasably fasten flexible rod 501 to pathfinder 60. For example, FIG. 13F shows a threaded type lock that is an example of one type of connector that could be used to releasably fasten flexible rod 501 to pathfinder 60. Release button 14 [seen in FIG. 14] is fastened to handle 11 such that as release button 14 is rotated, screw 67 also rotates and will screw into and out of a bore or opening 529 [seen in FIG. 13F]. Tongue 69 is a part of a “tongue-in-groove” type connection. This “tongue-in-groove” interconnection is clearly seen at FIG. 13F. The tongue 69 and groove 528 substantially prevent pathfinder 60 from rotating or spinning. Screw 67 is received by an opening 529. In the embodiment seen in FIG. 13F, opening 529 and screw 67 are threaded. As seen in FIG. 13F, the threaded portions ot screw 67 and threaded opening 529 mate rigidly to releasably fasten handle 11 to pathfinder 60. Collectively, tongue 69, groove 528, screw 67 and opening 529 substantially prevent flexible rod 501 from rotating relative to handle 11 and pathfinder 60. Movement of flexible rod 501 relative to handle 11 and pathfinder 60 is undesirable because it is more difficult for a surgeon to guide flexible rod 501 and through openings 44 of pedicle screws 40.

FIG. 13G shows another type of connector that could be used to releasably fasten flexible rod 501 and pathfinder 60. For example, a “snap-lock” type device 527 is appropriate. Snap-lock type devices 527 are well known and are used for the purpose of illustrating an alternative type connection for pathfinder 60 and flexible rod 501. Again, it is highly desirable to minimize or prevent flexible rod 501 from rotating or moving relative to handle 11.

FIG. 14 shows that different types of flexible rods 501 could be used in conjunction with pathfinder 60. In the embodiment seen in FIGS. 13A-13G and 14, handle 11 and pathfinder 60 serve as removable guides.

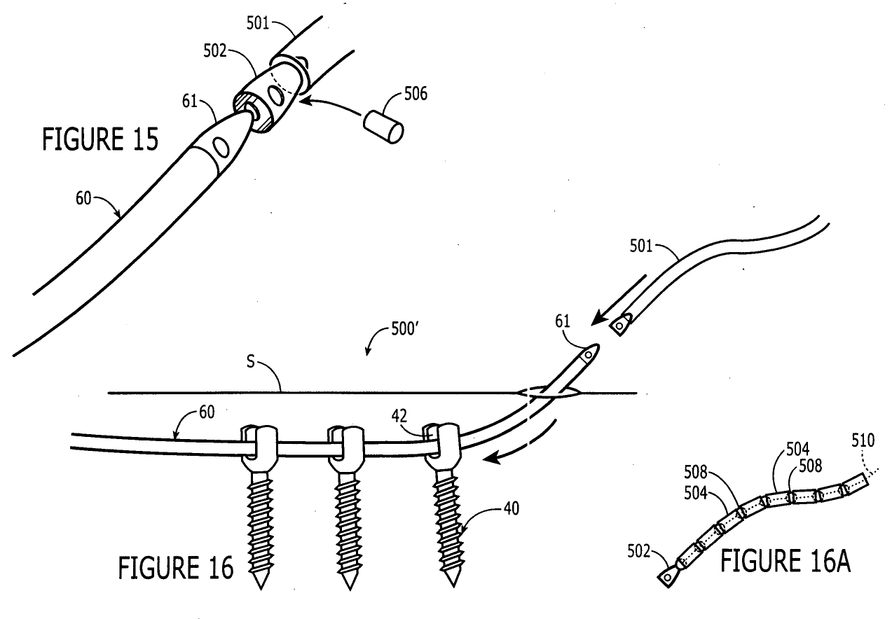

FIGS. 15, 16 and 16A illustrate an alternative retrograde rod delivery device 500′. Retrograde rod delivery device 500′ includes handle 11 (not shown), pathfinder 60 and flexible rod 501. Flexible rod 501 further includes connector 61 that is located at the distal end of pathfinder 60. Flexible rod 501 includes cap 502, links 504, pin 506, inserts 508 and stiffener 510. In its preferred use, seen in FIG. 16, pathfinder 60 is advanced through pedicle screws 40 using handle 11 (not shown). After pathfinder 60 is advanced through pedicle screws 40, connector 61 perforates the patients skin S, seen at FIG. 16, and the distal end of pathfinder 60 is located above the patient's skin S, cap 502 is mated to connector 61, best seen in FIGS. 15 and 16, and pin 506 is inserted to releasably fasten connector 61 and cap 502. After pathfinder 60 and flexible rod 501 are releasably connected, handle 11 is withdrawn, in the direction shown by the arrow in FIG. 16, and “drags” or pulls flexible rod 501 through channels 42 of pedicle screws 40. After flexible rod 501 is pulled through all the necessary channels 42 of pedicle screws 40, pin 506 is withdrawn and connector 61 disengaged from cap 502. Flexible rod 501 is then pulled tight, using tighter 510, to make flexible rod 501 substantially rigid such that pedicle screws 40 and flexible rod 501 act as a rigid internal splint to help immobilize and strengthen the spine during a period of bony healing. FIG. 16A shows that flexible rod 501 includes cap 502, links 504, inserts 508 and stiffener 510. When stiffener 510 is pulled tight, links 504 and inserts 508 are forced into close alignment and thereby prevent or minimize relative movement between links 504 and inserts 508. Because this relative movement is substantially prevented, flexible rod 501 effectively becomes substantially like an integral rigid rod.

It is within the scope of the present invention that flexible rod 501 could be a hollow tube and “cement” could be forced through the hollow tube to “harden” flexible rod 501 [seen FIGS. 13C, 13D and 13E]. It is also within the scope of the invention that alternative method of making rod 501 substantially rigid could be employed. An example of another alternative to “harden” flexible rod 501 would be a ferroelectric material that is pliable until exposed to electric current. Once exposed to an electric current, this ferroelectric material will harden to make rod 501 substantially rigid [seen at FIG. 13B and 14].

During the process seen in FIGS. 15, 16 and 16A, handle 11 may be advanced through channels 42 of pedicle screws 40 using any of the apparatus or methods disclosed above or any equivalent. In the embodiment seen in FIGS. 15, 16 and 16A, handle 11 and pathfinder 60 serve as removable guides.

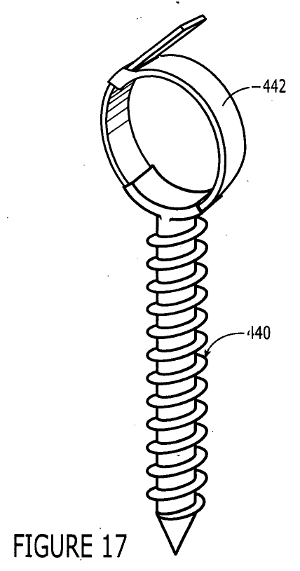

FIG. 17 illustrates an alternative pedicle screw 440 of the present invention. Alternative pedicle screw 440 provides a larger more forgiving target or loop 442 using “zip” technologies. Before insertion of rod 20, loop 442 may be wide open. After rod 20 is “lassoed,” loop 442 is pulled tight and collapses to tightly hold rod 20 to pedicle screw 440. Similar “collapsing target” screws are also within the scope of the present invention.

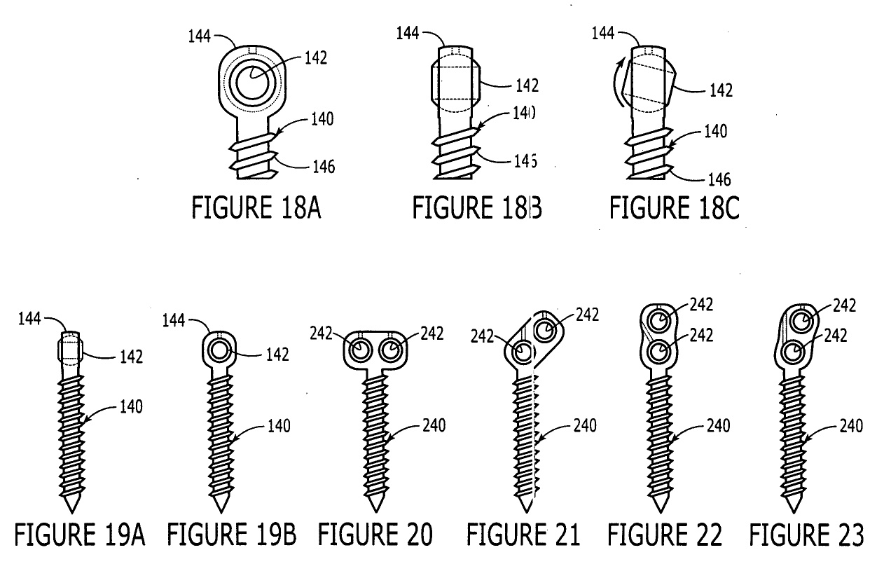

FIGS. 18A, 18B and 18C illustrate an embodiment of the adjustable uni-channel pedicle screw 140 of the present invention. Adjustable uni-channel pedicle screw 140 includes an adjustable channel 142, head 144 and screw portion 146. If a surgeon elects, adjustable uni-channel pedicle screw 140 may be used in conjunction with the elements seen in FIG. 1-16A, or other devices. As explained above, rod 20 passes through adjustable channel 142 to fasten two, or more, pedicle screws in rigid alignment. The “ball and socket” design seen in FIGS. 18A, 18B and 18C, could be replaced with any other type of structure that will allow adjustable channel 142 to move relative to pedicle screw head 144. The “ball and socket” design allows greater freedom of trajectory between points than a non-moveable/adjustable head for a pedicle screw. FIG. 18C particularly shows that channel 142 can be adjusted before or after inserting a pedicle screw. After rod implantation takes place, it desireable to “crimp” or otherwise prevent adjustable channel 142 from moving in order to hold rod 20 fixedly in place.

FIGS. 19A and 19B, illustrate an entire adjustable uni-channel pedicle screw 140 with an adjustable channel 142. As with the pedicle screws discussed above, adjustable uni-channel pedicle screw 140 is implanted in vertebrae and is below the surface of the patient's skin.

FIGS. 19A, 19B, 20, 21, 22 and 23, illustrate different embodiments of pedicle screws. FIGS. 11A, 11B, 11C, 11D, 12A and 12B illustrate the use of a five pedicle screws to immobilize and strengthen the spine during a period of bony healing. Typically, uni-channeled pedicle screws 40 or 140 will be the first and last in the series of pedicle screws used to rigidly fix pedicle screws and the spine in fixed alignment. In other words, uni-channeled pedicle screws 40 or 140 will be the rostral (closest to the head) and caudal (closest to the feet) pedicle screws in the series of pedicle screws used to rigidly fix pedicle screws and the spine in fixed alignment. A surgeon will use any of the devices or methods described above to place rods 20 between the pedicle screws.

Currently, multi-channeled pedicle screws 240, seen in FIGS. 20, 21, 22 and 23, are not known and a surgeon will make a single “pass” using a single rod to connect a series of pedicle screws by pushing rod 20 through channels of the pedicle screws. The multi-channeled pedicle screw 240 breaks this single “pass” into either multiple short passes or allows the surgeon to “steer” rod 20 through the pedicle screws 40, 140 and 240 more easily. The use of multi-channeled pedicle screws 240 allows a surgeon to make these passes either “free-hand,” “semi-free hand” or using the rod delivery devices described above. Among the benefits of a multi-channeled pedicle screw 240 is that two separate rods 20, with dramatically different trajectories, are connected to one pedicle screw 240. At the present time, when vertebrae are misaligned, it is very difficult to fasten a pedicle screw such that a surgeon can successfully pass a rod 20 through several single channeled pedicle screws. The present invention overcomes these difficulties by making the rod steerable and allowing a surgeon to position the pedicle screw such that it is easier to successfully pass a rod 20 through two, or potentially more, pedicle screws.

FIGS. 20, 21, 22 and 23, show that channels 242 may be side-by-side, or be displaced laterally or vertically or a combination depending on the type of anatomic offset required. In the preferred embodiment, a side-by-side arrangement [FIG. 20] is best for lateral offset, a “top-to-bottom”[FIG. 22] arrangement is best for vertical offset and a “domino” configuration [FIGS. 21 and 23] is best for maximum flexibility. Obviously, a surgeon would select a pedicle screw 240 such that rod 20 would interconnect with another pedicle screw. The positions of channels 242 are not limited to those seen in FIGS. 20, 21, 22 and 23, a surgeon could select a multi-channeled pedicle screw 240 with channels in any variety of positions required to best overcome the type of anatomic offset encountered.

A surgeon may use any of the devices or methods described above to place rods between any of the pedicle screws described above.

Methods of Pedicle Screw Selection

Pedicle screws 40 should be carefully selected according the diameter of the pedicle screw head 44, length of the pedicle screw and orientation of the pedicle screw head 44.

Pedicle screw diameter is preferably determined by the size of the pedicle as visualized on x-rays obtained in the operating room as well as through pre-operative imaging studies, including CAT scans and x-rays or other imaging techniques.

The length of the pedicle screw should be carefully selected to engage as much bony architecture, also known as bony vertebral elements, without being excessively long. An excessively long pedicle screw can potentially penetrate a patient's soft tissue elements. Imaging before the surgical procedure and x-rays taken in the operating room can be helpful in selecting the appropriate pedicle screw length.

The configuration of the pedicle screw head 44, relates to the degree of off-set in either the lateral or vertical dimension from an imaginary line connecting the pedicle screws at the terminal ends of the construct. For example, a pedicle screw construct containing four screws defines a line between the upper most and lower most screw might vary significantly with regard to laterality or superior, inferior orientation of the screw relative to the imaginary line between the first and last screws of the construct. If a screw in the interval between the upper most and lower most pedicle screws were to be 15 mm to the right of the line and the screw next to it 15 mm to the left of the imaginary line, interconnecting these pedicle screw could prove very difficult and use of a multi-channeled pedicle screw 240 could neutralize the offset of the intervening pedicle screws by allowing the pedicle screws heads 244 to minimize the distance from the imaginary line. The advantage of the multi-channeled pedicle screw would be that rather than having to transverse widely divergent points with a single rod, the course of the rod could be “broken” or divided into several smaller distances allowing easier angulation from one pedicle screw head 44 to the next.

Method of Pedicle Screw Placement

Typically, after exposing the surgical area, the next step is placement of pedicle screws into the vertebral elements. Typically, the areas where the surgeon would like to place pedicle screws are visualized by x-ray. Using a small needle, and the guidance of the x-ray, the needle is pushed through the skin to the area of desired entry for the pedicle screw into the bony vertebral elements. After the surgeon confirms the path, also known as a trajectory, through the patient's skin to achieve satisfactory and safe placement of the pedicle screw, a small skin incision is made on the patient's skin surface. After the small skin incision is made, several methods can be used to place the pedicle screw in the bony elements of the patient's spine. One method involves cannulation of the bone using a sturdy hollow needle, which is driven under x-ray guidance into the bone allowing for placement of a guiding wire into the bony vertebral elements. A cannulated tap can be inserted over the wire, carefully following the trajectory of the wire as the tap is advanced. Following withdrawal of the tap, the pedicle screw, which is itself cannulated, can be advanced with a hollow screwdriver allowing the pedicle screw to placed over the guide wire along a previously tapped trajectory. Another method of placing a pedicle screw into bony vertebral elements involves placing a small profile, thin small diameter retractor directly onto the bone surface through the skin incision. This can be step can follow the use of direct visualization of the bony elements. A device to palpate, or “feel,” along the inner surface of the desired bone trajectory can also be inserted. A tap could also follow this process. Following these steps, the pedicle screw is placed into the bony vertebral elements. In each of these techniques, the liberal use of x-ray techniques is appropriate to facilitate safe placement of the pedicle screws into solid bony vertebral elements and also to avoid neural and soft tissue elements.

Multiple small perforations of the patient's skin at appropriate intervals along the patient's spine allow a surgeon to place additional pedicle screws, or other fixation devices, at various intervals along the patient's spine.

After the necessary pedicle screws, or other appropriate fixation devices, are successfully inserted into the bony vertebral elements, the pedicle screws should be interconnected to successfully restore structural integrity of the patient's spine. This method of interconnecting the pedicle screws using rods is referred to as the “Method of Placing Rods Using Rod Delivery Device” or “Rod Delivery Method.”

Methods of Placing Rods Using Rod Delivery Device

The patient is positioned prone, also known as “face down,” on an operating room bed that is preferably radiolucent, such that a surgeon can employ x-ray imaging during the operative procedure to locate and visualize bony landmarks of the spine.

It is desirable to visualize bony landmarks pre-operatively using x-rays to enhance safe placement of pedicle screws. Because percutaeous procedures are, by definition, performed below the patient's skin, x-rays are also useful in confirming the interconnective relationship between the rods and the pedicle screws as they are mated during the surgical procedure.

Typically, the patient undergoes a through cleaning of the area of the operative procedure and placement of surgical drapes to isolate the operative area from contamination.

Typically, the surgeon will already have selected the appropriate pedicle screws necessary for the procedure. The methods of pedicle screw selection are discussed earlier in this document and need not be repeated here.

Typically, the surgeon will then place the pedicle screws into the vertebral elements using the methods discussed above. One of the primary advantages of the present rod delivery device and method is that the surgeon can affirmatively choose to place the pedicle screws at optimal positions in the vertebral bone to minimize potential contact with neural structures or soft tissue, as opposed to modifying his pedicle screw position in order to maximize co-linearity of the pedicle screws with one another.

After selection of the pedicle screws and placement of the pedicle screws, the surgeon's task is to interconnect the pedicle screws utilizing at least one of the rod delivery devices. The methods of pedicle screw interconnection with a rod can vary depending on a surgeon's personal preference, the surgical equipment available or the surgeon's personal choice. For example, the methods of using the rod delivery device fall into six types. First, a “free-hand” rod delivery method. Second, “bayonet” rod delivery method. Third, using a “neuronavigational” system rod delivery method. Fourth, using a “retrograde” rod delivery method. Fifth, a steerable rod device method. Each of these methods will be discussed in turn.

1) Free Hand Rod Delivery Method

The free hand rod delivery method may be used after all pedicle screws are placed, or alternatively, a surgeon could place two pedicle screws and then interconnect them using a rod and then repeat this process. Typically, it is recommended that all pedicle screw be placed before the interconnection process begins.

While the present method description typically refers only to uni-channel pedicle screws 40, it is with in the scope of the present invention to use a multi-channeled pedicle screw [for example, as seen in FIGS. 20-23], an adjustable uni-channeled pedicle screw [for example, as seen in FIGS. 18A, 18B and 18C], an adjustable multi-channeled pedicle screw [for example, as seen in FIGS. 20-23], or a loop pedicle screw [as seen in FIG. 17] for the any of the methods of rod delivery.

FIGS. 1 and 5 shows a handle 11, shaft 16 and rod 20. A surgeon may chose to exchange any of these pieces for an alternative piece that is more appropriate for the patient's body type and vertebral placement. Typically, a surgeon will initially select a handle 11. It is important that handle 11 is appropriate. For example, a handle that hinges inferiorly (i.e. below) from the axis of the rod 20 may abut the patient's skin surface as the rod 20 is advanced. A handle that extends superior (i.e. above) the axis of the rod 20 may allow rod manipulation without abutting the patient's skin surface. Some trial and error may be required to choose the appropriate handle shape, contour and grip 12's configuration.

Typically, the surgeon will then select a shaft 16. Alternatively, as seen in FIG. 5, the surgeon might also select an alternative shaft 116. Of course, a surgeon is not limited to a single alternative shaft. As discussed earlier in this document, a surgeon could select an alternative shaft based on a number of criteria. Typically, a surgeon will select an alternative shaft because the surgeon must avoid an adjacent bony prominence or because the trajectory to the first pedicle screw is shallow or steep. It is within the scope of the present invention that a surgeon will use alternative shafts under different surgical circumstances.

Typically, the surgeon will then select an appropriate rod 20 to interconnect the pedicle screws 40. As seen in FIG. 6, preferably, the surgeon should consider the length of the rod required. Preferably, rod 20 will extend just a few millimeters beyond the pedicle screw 40 to allow adequate fixation of the pedicle screw 40 to the rod 20 without too much “overhang.” If there is excessive “overhang,” rod 20 may bind on surrounding soft tissues or abut other bony elements. Excessive “overhang” is typically considered undesirable.

FIGS. 6A and 6B show that a surgeon must also consider the diameter of pedicle screw 40. Typically, a surgeon will anticipate varying diameters for rods 20 based on the application and stresses that might be encountered or anticipated. A rod 20 with a smaller diameter might be used in the upper, also known as cervical, spine, while larger diameter rods 20 would mate to larger diameter pedicle screw channel's 42. It is undesirable to use a rod 20 with a diameter that is substantially different than the diameter of pedicle screw channel 42. As also seen in FIGS. 6A and 6B, it is desirable to select a rod 20 that mirrors the geometry of the section of spine between the pedicle screws that the rod 20 is interconnecting.

FIG. 6 shows that the curvature of the rod 20 should mirror the physiologic curves of the spine. For example, a surgeon might use alternative rod 220 in sections of the lumbar spine because this section of the human spine typically has a concave curvature. Areas of the thoracic spine typically have a convex curvature. As seen in FIG. 6, alternative rod 620, or another generally convex alternative rod, would best mirror this curvature. Of course, a surgeon could select an alternative handle and an alternative shaft to use in conjunction with alternative rod 620 or 220.

Free hand placement of the rod 20 into the pedicle screw 40 should begin with close assessment of the x-ray images obtained in the operating room. Preferably, the surgeon should obtain images in antero/postero and lateral planes. The ability to adequately visualize the “target” of the rod 20, namely the where the rod 20 will engage the second pedicle screw 40 in the series, is important to achieve appropriate mating of the rod 20 with the pedicle screw 40. Using radio opaque markers may assist in determining an approximate trajectory for the rod delivery device 10 and the trajectory could be marked out and superimposed onto the skin surface.

The surgeon should next make a small skin incision [for example, as seen in FIG. 13A] and the rod delivery device 10 could be advanced using direct x-ray guidance to gently advance the rod delivery device 10 through the soft tissues to positively engage pedicle screw 40. The surgeon should also take care that the tip of the rod is suitably positioned such that rod will smoothly transition toward the next fixation point, i.e. the next pedicle screw 40. Examples of taking care that the rod should exit the first pedicle screw 40 in the series such that the tip of is positioned to smoothly transition toward the next pedicle screw can be seen in FIGS. 6A and 6B. The free hand rod delivery method might allow the placement of a single rod 20 through two, three, four or more pedicle screws, such as seen in FIGS. 6, 6A and 6b.

It is also possible that it might be advantageous to “break up” the trajectory into several smaller passes using multi-headed pedicle screws 240. FIG. 11A shows the using rod delivery device 10 to interconnect pedicle screw 40 and multi-headed pedicle screw 240. In another words, a single rod 20 is used to interconnect pedicle screw 40 and multi-headed pedicle screw 240 seen in FIG. 11 Å. Once rod 20 has been delivered through the desired trajectory and engaged at least two pedicle screws, in the example seen in FIG. 11A pedicle screws 40 and 240, the surgeon should undertake an assessment of the length of rod 20 to determine that rod 20 is neither too long nor too short for the application. Typically, this length assessment is conducting using x-ray guidance. After determining that rod 20's length is appropriate, rod 20 should be positively engaged to both pedicle screws 40 and 240 by using tighteners T [not shown]. Typically, tightener T [not shown] is located above the skin S. Once the surgeon has satisfactorily secured rod 20 to each of the pedicle screws 40 and 240, the handle 11 could be withdrawn and the next rod selected for delivery. This process is repeated until each pedicle screw is interconnected with the pedicle screw before it in the sequence. Typically, it is not necessary to use a multi-headed pedicle screw 240 for either the first or last pedicle screw in the series. Tightener T are well known by surgeons and are not shown in FIGS. 11A, 11B, 11C or 11D.

2) Bayonet Rod Delivery Method

Another method to facilitate the placement of a rod into a series of pedicle screws, while minimizing the amount of intra-operative x-ray that might be required is to use a bayonet rod delivery method. In this method, seen at FIGS. 7, 7A, 7B and 8, alternative rod delivery system 100, handle 11, rod 20 and bayonet attachment 30 allow rod 20 to interconnect two pedicle screws 40 while minimizing intra-operative x-ray use. Handle 11 is used to maneuver rod 20 through two or more pedicle screws 40. Bayonet attachment 30, in cooperation with pedicle screw extenders 50, assist the surgeon in guiding rod 20 through channels 42 of pedicle screws 40 [best seen in FIG. 8].

As also seen in FIGS. 7, 7A, 7B and 8, screw extenders 50 act as guidance phantoms and also allow dynamic forces to be placed on the spine during insertion and tightening. As seen in FIG. 7, pedicle screw 40 is inserted posteriorly into the thoracic or lumbar spine. Screw extender 50 is removably fastened to pedicle screw 40. Because pedicle screw 40 is implanted into a vertebra, it is below the surface of the patient's skin S. Screw extender 50 extends from the top 44 of pedicle screw 40, through the patient's skin, and is exposed to the surgeon above the patient's back. Head 52, of screw extender 50, includes notch 54 and groove 56. Notch 54 and groove 56 slidably receive bayonet attachment 30 and ridge 34. In a preferred embodiment, seen in FIGS. 7, 7A and 8, ridge 34 is slidably received by notch 54. Cooperatively, ridge 34 and notch 54 prevent bayonet attachment 30 from rotating relative to head 52 of screw extender 50. Effectively, bayonet attachment 30 should be cruciate, so as to allow control of alternative rod delivery device 100 in multiple planes. It should also be apparent that screw extender 50's head 52 is an above skin phantom that is used to guide rod 20 through channels 42 of pedicle screws 40.

Because rod 20 and bayonet attachment 30 move in tandem, when a surgeon guides bayonet attachment 30 through groove 56 and notch 54, rod 20 passes through channel 42 of pedicle screw 40.

Once the approximate path of rod 20's fixation has been determined using pedicle screw extensions 50, the surgeon should make a small incision in the patient's skin allowing the surgeon to deliver rod 20 using rod delivery device 100 to the first pedicle screw 40. The surgeon will use the visual cues provided by the above skin portion of pedicle screw extender 50 to guide rod 20's placement. This process could be continued from pedicle screw to pedicle screw as required or could be employed simply as an initial docking method. If the surgeon chooses, other delivery methods could be employed to connect the second and subsequent pedicle screws.

3) Neuronavigational System Rod Delivery Method

Another method of interconnecting pedicle screws is to use neuronavigational techniques. As seen in FIGS. 9, 9A, 9B and 9C, neuronavigational techniques use sophisticated computer technology to allow a surgeon to know precisely where an object in space is located with respect to a patient s anatomy.

As discussed earlier, neuronavigational systems, such as neuronavigational system 210, for spine and brain surgery are known and regularly used. For example, as disclosed at U.S. Pat. No. 5,383,454, issued to Buchholz, on Jan. 24, 1995, for system for indicating the position of a surgical probe within a head on an image of the head and at U.S. Pat. No. 6,236,875, issued to Buchholz, on May 22, 2001, for surgical navigation systems including reference and localization frames. In other words, those of skill in the art know neuronavigational systems. However, those of skill in the art have not used neuronavigational systems to interconnect pedicle screws using rods.

Alternative rod delivery system 200 includes handle 11, shaft 16, rod 20, pedicle screws 40, screw extenders 50 and a neuronavigational system 210. Neuronavigational system 210 uses detectional spheres 230 and 231, comparator 235 and display 238.

Preferably, detectional spheres 231 are positioned on the head 52 of each screw extender 50 and detectional sphere 230 is positioned proximate to handle 11. It is important that detectional spheres 231 are fixedly positioned relative to screw extenders 50. It is also desirable that detectional sphere 230 remains in the same relative position to handle 11. If the detectional spheres do not remain fixed relative to the structures they are associated with, the neuronavigational system cannot guide rod 20 through channel 42 of pedicle screw 40. Comparator 235 calculates the relative positions of handle 11, shaft 16, rod 20 and channels 42 of pedicle screw 40 because the relative positions of detector spheres 230 and 231 are known. Because comparator 235 “detects” the relative positions of handle 11, shaft 16, rod 20 and channel 42 of pedicle screw 40, display 238 visually displays this information. Information seen on display 238 indicates which direction a surgeon should move tip 26 of rod 20 to pass through the channels 42 of pedicle screws 40. Other than directional spheres 230 and 231, comparator 235 and display 238, the neuronavigational system 210 is not shown.

FIG. 9D shows the method of using the neuronavigational system rod delivery method. As discussed above, the surgeon selects an appropriate handle 11, shaft 16 and rod 20. After selecting and placing the pedicle screws 40 using the methods discussed above, the surgeon should make a small incision in the skin allowing the surgeon to deliver rod 20 using alternative rod delivery device 200 to the first pedicle screw 40. The surgeon will use the information provided by neuronavigational system 210 to guide rod 20's placement. This process could be continued from pedicle screw to pedicle screw as required or could be employed simply as an initial docking method. If the surgeon chooses, other delivery methods could be employed to connect the second and subsequent pedicle screws.

4) Retrograde Rod Delivery Method

FIGS. 13A, 13B, 13C, 13D, 13E, 13F, 13G and 14, show the retrograde rod delivery method using retrograde rod delivery device 500. After selecting and placing pedicle screws 40 using the methods discussed above, the surgeon should make a small incision in the skin S allowing the surgeon to deliver pathfinder 60.

The surgeon should select pathfinder 60 using similar considerations given to selecting shaft 16 and rod 20 of the earlier described methods. In other words, the surgeon should consider the length of pathfinder 60 required. A surgeon should also consider the diameter of pedicle screw 40's channel 42. It is undesirable to use a pathfinder 60 with a diameter that is substantially different than the diameter of pedicle screw channel 42. As also seen in FIG. 13B, it is desirable to select a pathfinder 60 that mirrors the geometry of the section of spine between the pedicle screws implanted by the surgeon. In other words, it is desirable that the curvature of the pathfinder 60 should mirror the physiologic curves of the spine.

FIG. 13A shows pathfinder 60 passing through the incision and then through channel 42 of first pedicle screw 40 of the three shown. FIGS. 13A-13G and 14 show three pedicle screws 40. However, the present method could be used for any number of pedicle screws as a surgeon may choose to employ. Retrograde rod delivery device 500 should be advance carefully through channels 42 of pedicle screws 40 until the distal tip of pathfinder 60 extends above the patient's skin S.

FIG. 13B shows retrograde rod delivery device 500 after it has passed through the three pedicle screws 40 implanted by the surgeon. After exiting channel 42 of the last pedicle screw 40 in the series, the surgeon should gently force the distal tip of pathfinder 60 out through the skin S. Flexible rod 501 should then be attached to the distal end of pathfinder 60 that is protruding through skin S. It is within the scope of the invention that flexible rod 501 could be an hollow hardening tube, a non-rigid memory metal, a flexible rod formed from ferroelectric material that is pliable until exposed to electric current or a locking rod and ball system or other equivalent flexible rods that can become stiff on demand.

The surgeon should select flexible rod 501 using similar considerations given to selecting shaft 16 and rod 20 of the earlier described methods. In other words, the surgeon should, at a minimum consider the length of flexible rod 501 required. A surgeon should also consider the diameter of pedicle screw 40's channel 42. It is undesirable to use a flexible rod 501 with a diameter that is substantially different than the diameter of pedicle screw channel 42.

FIGS. 13F and 13G show two of the numerous ways that flexible rod 501 and pathfinder 60 could be connected and disconnected. For example, FIG. 13F shows a “tongue-in-groove” type connection. Tongue 69 and groove 528 minimize flexible rod 501's from rotation or spinning. Pathfinder 60 is received by an opening 529. In the embodiment seen in FIG. 13F, opening 529 and pathfinder 60 are threaded. As seen in FIG. 13F, threaded screw 67 of pathfinder 60 and threaded opening 529 mate rigidly to releasably fasten pathfinder 60 to flexible rod 501. Collectively, tongue 69, groove 528, and opening 529 substantially prevent flexible rod 501 from rotating relative to pathfinder 60.

As shown in FIG. 13G, handle 11 can use a number of connections to fasten to pathfinder 60 to flexible rod 501. For example, a “snap-lock” type device 527 is appropriate. Snap-lock type devices 527 are well known and are used for the purpose of illustrating an alternative type connection for pathfinder 60 and flexible rod 501. It is also important that the surgeon can readily disconnect pathfinder 60 and flexible rod 501. It is also within the scope of the present invention that a surgeon could use a snap collar [not shown], a pin [shown at FIG. 16] or an internal expansion device [not shown], or any other equivalent interconnection device with any of the rod delivery devices or methods.

FIG. 13D shows the surgeon withdrawing pathfinder 60 in the direction of the arrow. After the surgeon has carefully withdrawn the pathfinder 60 through the incision, the surgeon should carefully disconnect pathfinder 60 from flexible rod 501. At this point, the surgeon should stiffen flexible rod 501. Depending on the type of flexible rod 501 in use, this stiffening could be accomplished by injecting core material into flexible rod 501, as seen in FIG. 13D.

It is within the scope of the present invention that flexible rod 501 could be a hollow tube and “cement” could be forced through the hollow tube to “harden” flexible rod 501 [seen FIGS. 13C, 13D and 13E]. It is also within the scope of the invention that alternative method of making rod 501 substantially rigid could be employed. An example of another alternative to “harden” flexible rod 501 would be a ferroelectric material that is pliable until exposed to electric current [FIG. 14]. Once exposed to an electric current, this ferroelectric material will harden to make rod 501 substantially rigid [seen at FIG. 14].

Obviously, the appropriate length for the flexible rod 501 would be gauged before selecting insertion. The appropriate length is just slightly beyond the terminal lengths of the most rostral and most caudal pedicle screws. In addition, when using the retrograde rod delivery method, it is desirable to engage the central pedicle screw before making flexible rod 501 rigid. By tightening only the central pedicle screw, this would allow flexibility in the other screws and would make it easier for the surgeon to bring flexible rod 501 and pedicle screws 40 into the most appropriate alignment. It is also preferable to tighten the non-central pedicle screws after flexible rod 501 is made rigid.

After flexible rod 501 is made rigid, any apparatus used to make flexible rod 501 rigid should be disconnected and removed as seen in FIG. 13E.

While only uni-channeled pedicle screws 40 are shown in conjunction with the retrograde rod delivery method, it is within the scope of the present invention to use uni-channeled pedicle screws 40, multi-channeled pedicle screws 240 or a combination of these two types of pedicle screws. In addition, FIGS. 13A, 13B, 13C, 13D, 13E, 13F, 13G, 14, 15, 16 and 16A show the retrograde rod delivery method delivering a flexible rod 501 in a single “pass.” However, it is within the scope of the present invention that a long series of pedicle screws could be interconnected with a series of passes.

5) Steerable Rod Device Method

FIG. 10 illustrates another alternative embodiment of a rod delivery device. Steerable rod delivery system 300 includes handle 11, steering mechanism 310, rod 20, steerable rod tip 312, pedicle screw 40 and pedicle screw channel 42. A steerable rod tip 312 is fastened to the distal end 26 of rod 20. Steering wire 314 may be a wire, or other similar structure, that can guide steerable tip 312. Steerable rod delivery system 300 guides rod 20 through one or more of pedicle screw 40's channels 42.

Steerable devices, and particularly steerable catheters, are known to those skilled in the art. An example of a steerable device is a “shapeable handle for steerable electrode catheter” that is disclosed at U.S. Pat. No. 5,397,304, issued to Truckai, on Mar. 14, 1995.

Because every surgeon has encountered a situation where the rod 20 is “just off,” it is advantageous to be able to manipulate the distal end of rod 20 to maneuver rod 20 through the channel 42 of pedicle screw 40. As discussed above, when a surgeon is “just off,” it is desirable to able to manipulate the distal end of rod 20 once the surgeon discovers, by use of x-ray, neuronavigational system or other visualization techniques, that the distal end of rod 20 is “just off.” As seen in FIG. 10, steerable rod delivery system 300 can slightly adjust the position of the tip/distal end 26 of rod 20 using a method of internal tensioning wires, articulating rods or electromechanical benders. It is also within the scope of the present invention that other methods, such as an articulation or a steerage mechanism between the terminal end of shaft 16 and the proximal end of rod 20, could be used. In other words, a steerable rod device uses a “pivot point” located between shaft 16 and rod 20. If the tip/distal end 26 of rod 20 was “just off,” the pivot point could be electronically commanded, either by means of a wire passing through shaft 16 or remotely, to slightly move in the desired direction. Us of a “pivot point” would eliminate the need for a complicated mechanism traveling through rod 20 itself.

### SUMMARY OF METHODS

The five methods set forth above each may incorporate the following steps:
1) the patient is positioned prone/face down on a radiolucent operating room table;
2) liberal use of intra-operative x-rays, and particularly fluoroscopic imaging to allow real time assessment of bony elements;
3) selection of appropriate type and number of pedicle screws;
4) placement of pedicle screws into bone using a system of placement of cannulated screws over a wire and direct visualization of the bony elements with small retractors;
5) selection of a handle of appropriate size and shape to accommodate the physical contours of the patient;
6) selection of a shaft of appropriate contour to accommodate the physical contours of the patient;
7) selection of a rod of appropriate contour to accommodate the physical contours of the patient;
8) “threading” the rod into the channels of the pedicle screws placed into the patient's bone using a single pass, multiple single passes or one or more multiple passes;
9) positively engaging pedicle screws to rod or rods; and,
10) closing the patient's wounds.

The above method would change if a surgeon used the retrograde rod delivery method or steerable rod method. For the retrograde method, the apparatus for placing the rod would have to be withdraw and any additional apparatus for making the flexible rod rigid would have to be introduced and then withdrawn after the flexible rod is made rigid. With respect to the steerable rod delivery method, the step of “threading” the rod into the channels could include “steering the rod tip” to urge the tip through the channel of the pedicle screw in question. In addition, it is also possible to use a steerage rod delivery system in combination with the retrograde rod delivery method.

Surgical Kits

The following items might be included in a surgical kit provided to a surgeon performing percutaneous rod implant in a human spine.
A variety of handles of different shapes and geometries;
A variety of handles of different lengths;
A variety of handles of different curvatures;
A variety of handle grips, including grips that are primarily above the access of the handle and grips primarily below the access of the handle.
A variety of shafts of different lengths.
A variety of shafts of different curvatures.
A variety of shafts of different diameter based on pedicle screw channel widths likely needed for the present operation.
   Rods
A variety of rods of different lengths.
A variety of rods of different diameters, based on pedicle screw orifice sizes.
A variety of rods of various curvatures.
   Steerable Rod Drivers
A steerable rod driver with steerable terminal articulation and steerable articulation of handle and rod interface.
A steerable mechanism without rod adaptor.
   Pedicle Screws
Pedicle screws of conventional type.
Pedicle screws of multiple head type.
Pedicle screw types of multiple diameters, and multiple lengths.
Bayonet attachment for handle.
Attachment for neuronavigation devices, also known as detectional spheres.
Pedicle screw extenders.
Fixed reference device for rigid fixation to spine.
Pedicle screw extenders for bayonet engagement.
Pedicle screw extension with adaptors for neuronavigational use.
Rod benders to custom configure rods if not to optimal contour.
Rod cutters to customize rod length.
Fixation screwdrivers to engage the pedicle screw through the small soft tissue defect/skin incision above the pedicle screw.
Thin gauge wire for determining optimal point of skin incision and trajectory for pedicle screw fixation.
Small retractors to allow direct visualization of pedicle screw entry point.
Surgical air drill to allow decortications of bony pedicle screw entry point.
Miscellaneous extras of small components that may be lost or misplaced at the time of surgery.
Sterilization boxes for instruments.
Packing lists for boxes.
Mailing forms

While the invention has been illustrated and described in detail in the drawings and description, the same is to be considered as an illustration and is not limited to the exact embodiments shown and described. All equivalents, changes and modifications that come within the spirit of the invention are also protected by the claims that are set forth below.

1. A minimally invasive spinal fixation system used for spinal arthrodesis or motion preservation spinal repair, comprising:
a plurality of pedicle screws, including a first screw placed into a first vertebral body, and a second screw placed into a second vertebral body;
an attachment assembly for connecting said pedicle screws, said assembly comprising:
a connector for attaching to said first screw and said second screw;
a removable guide for percutaneously attaching the connector to said first screw and said second screw.

2. A minimally invasive spinal fixation system as in claim 1, wherein said connector is a rod.

3. A minimally invasive spinal fixation system as in claim 1, wherein said connector is a plate.

4. A minimally invasive spinal fixation system as in claim 1, wherein said connector is a pin.

5. A minimally invasive spinal fixation system as in claim 1, wherein said connector is a flexible rod.

6. A minimally invasive spinal fixation system as in claim 5, wherein said flexible connector is a polymer fillable rod.

7. A minimally invasive spinal fixation system as in claim 5, wherein said flexible connector is a flexible rod with a link and an insert, whereby a stiffener can force said link and said insert into close alignment and thereby prevent or minimize relative movement between said link and said insert.

8. A minimally invasive spinal fixation system as in claim 5, wherein said flexible connector is a cement fillable-to-harden rod.

9. A minimally invasive spinal fixation system as in claim 5, wherein said flexible connector is formed of ferroelectric material that is pliable until exposed to electric current.

10. A minimally invasive spinal fixation system as in claim 5, wherein said flexible connector is a thin rod and whereby there is some preservation of motion.

11. A minimally invasive spinal fixation system as in claim 5, wherein said flexible connector is a polymer rod and whereby there is some preservation of vertebral motion.

12. A minimally invasive spinal fixation system as in claim 1, wherein at least one of said pedicle screws is a uni-channeled pedicle screw.

13. A minimally invasive spinal fixation system as in claim 12, wherein said uni-channeled pedicle screw further comprises an adjustable channel.

14. A minimally invasive spinal fixation system as in claim 1, wherein at least one of said pedicle screws is a multi-channeled pedicle screw.

15. A minimally invasive spinal fixation system as in claim 14, wherein said multi-channeled pedicle screws further comprises an adjustable channel.

16. A minimally invasive spinal fixation system as in claim 1, wherein said removable guide is a handle.

17. A minimally invasive spinal fixation system as in claim 1, wherein said removable guide is a handle and a shaft.

18. A minimally invasive spinal fixation system as in claim 1, wherein said removable guide comprises a handle, a shaft and a bayonet attachment.

19. A minimally invasive spinal fixation system as in claim 1, wherein said removable guide comprises a handle, a shaft and a computer aided surgery device.

20. A minimally invasive spinal fixation system as in claim 1, wherein said removable guide comprises a handle, a steering mechanism and a steerable tip.

21. A minimally invasive spinal fixation system as in claim 1, wherein said removable guide comprises a handle and a pathfinder.

22. A minimally invasive spinal fixation system as in claim 19, wherein said computer aided surgery device is a neuronavigational system.

23. A rod delivery device, comprising:
a handle;
a rod releasably fastened to said handle; and,
a pedicle screw having a channel there through;
whereby said handle allows a surgeon to guide said rod through said channel in said pedicle screw using said handle.

24. A rod delivery device, comprising:
a handle;
a bayonet attachment fastened to said handle;
a rod releasably fastened to said rod handle;
a pedicle screw having a channel there through; and
a screw extender fastened to said pedicle screw;
whereby said bayonet attachment and said screw extenders act as guidance phantoms to assist a surgeon in guiding said rod through a channel in said pedicle screw.

25. A multi-channeled pedicle screw, comprising:
a screw portion;
a head fastened to said screw portion; and,
a plurality of channels disposed there through said head wherein the locations of said plurality of channels are selected based on the locations best sited to best overcome the type of anatomic offset required.

26. A minimally invasive method for using pedicle screws to stabilize vertebral bodies anatomically positioned in a patient, the method comprising:
percutaneously placing a first pedicle screw into a first vertebral body and second pedicle screw into a second vertebral body;
percutaneously inserting a connector into the patient into a first position adjacent the first pedicle screw, the connector designed to accommodate the anatomical positions of the vertebral bodies and the orientation of said first pedicle screw and said second pedicle screw;
guiding the connector from said first position to a second position adjacent said second pedicle screw;
attaching said connector to the first pedicle screw and the second pedicle screw.

27. A minimally invasive method of using pedicle screws to stabilize vertebral bodies as in claim 26, wherein said step of guiding the connector from said first position to said second position adjacent to said second pedicle screw comprises:
selecting a suitable handle; and,
releasably fastening said handle to said connector;
wherein said handle is used to guide said connector from said first position to a second position adjacent said second pedicle screw.

28. A minimally invasive method of using pedicle screws to stabilize vertebral bodies as in claim 26, wherein said step of guiding the connector from said first position to said second position adjacent to said second pedicle screw comprises:
selecting a suitable handle;
selecting a suitable bayonet attachment;
fastening a screw extender to said pedicle screws, said pedicle screw having a channel there through, and said screw extender having groove there through; and,
passing said connector through said channel using said bayonet attachment as a guidance phantom;
wherein said connector acts as an internal splint to immobilize and strengthen the spine during a period of bony healing.

29. A minimally invasive method of using pedicle screws to stabilize vertebral bodies as in claim 26 wherein said step of guiding the connector from said first position to said second position adjacent to said second pedicle screw comprises:
selecting a suitable handle;
selecting a suitable shaft; and,
selecting a suitable computer aided surgery device;
guiding said connector from said first position to a second position adjacent said second pedicle screw using said handle, said shaft and said computer aided surgery device.

30. A minimally invasive method of using pedicle screws to stabilize vertebral bodies as in claim 29, wherein said computer aided surgery device is a neuronavigational system.

31. A minimally invasive method of using pedicle screws to stabilize vertebral bodies as in claim 26, wherein said step of guiding the connector from said first position to said second position adjacent to said second pedicle screw comprises:
selecting a suitable handle;
selecting a suitable steering mechanism;
selecting a suitable steerable tip;
wherein said handle, said steering mechanism and said steerable tip guide said connector from said first position to a second position adjacent said second pedicle screw.

32. A minimally invasive method of using pedicle screws to stabilize vertebral bodies as in claim 26, wherein said step of guiding the connector from said first position to said second position adjacent to said second pedicle screw comprises:
selecting a suitable handle;
selecting a suitable pathfinder;
passing said pathfinder through a channel of a pedicle screw;
fastening said connector to said pathfinder;
drawing said connector through said channel of said pedicle screw; and,
hardening said connector such that said connector becomes substantially rigid;
whereby said connector acts as an internal splint to immobilize and strengthen the spine during a period of bony healing.

33. A minimally invasive method of using pedicle screws to stabilize vertebral bodies as in claim 32 wherein said step of fastening said connector to said pathfinder comprises using a tongue-in-groove connection.

34. A minimally invasive method of using pedicle screws to stabilize vertebral bodies as in claim 32 wherein said step of fastening said connector to said pathfinder comprises using a snap-lock connection.

35. A surgical kit used for minimally invasive spinal arthrodesis or motion preservation spinal repair, the kit comprising:
a plurality of pedicle screws;
a plurality of connectors;
a guide comprising a handle and a plurality of removable shafts attachable to said connectors, said shafts designed to connect to one or more of said connectors.

36. A surgical kit as in claim 35, wherein said pedicle screws are selected from the group consisting of uni-channeled pedicle screws, multi-channeled pedicle screws, uni-channeled pedicle screws with an adjustable channel, multi-channeled pedicle screws with an adjustable channel and pedicle screws with a loop.

37. A surgical kit as in claim 35, wherein said connectors are selected from the group consisting of rods, plates, pins, flexible rods, polymer fillable rods, flexible rods and flexible connectors.

38. A surgical kit as in claim 37, wherein said flexible rods are selected from the group consisting of polymer fillable rods, flexible rods with a link and an insert, cement fillable-to-harden rods, flexible rods formed of ferroelectric material that is pliable until exposed to electric current, thin rods and polymer rods.

39. A surgical kit as in claim 35, wherein said guide is a handle.

40. A surgical kit as in claim 35, wherein said guide is a handle and a shaft.

41. A surgical kit as in claim 35, wherein said guide is a handle, a shaft and a bayonet attachment.

42. A surgical kit as in claim 35, wherein said guide is a handle, a shaft and a computer aided surgery device.

43. A surgical kit as in claim 42, wherein said computer aided surgery device is a neuronavigational system.

44. A surgical kit as in claim 35, wherein said guide is a handle, a steering mechanism and a steerable tip.

45. A surgical kit as in claim 35, wherein said guide is a handle, a pathfinder and a flexible rod.
